SEQUENCE LISTING (209 sequences)

SEQ ID NO: 1 - RNA, 86 nt, Homo sapiens
cuaccccaaaauaugguacccuggcauacugaguauuuuaauacuggcauacucaguaugccauguugccauauuuugggguagca

SEQ ID NO: 2 - RNA, 88 nt, Homo sapiens
uuugcauuaaaaaugaggccuucucuucccaguucuucccagagucaggaaaagcuggguugagaggguagaaaaaaaaugauguagg

SEQ ID NO: 3 - RNA, 66 nt, Homo sapiens
agcagcccucggcggcccggggggcgggcggcggugcccgucccggggcugcgcgaggcacaggcg

SEQ ID NO: 4 - RNA, 75 nt, Homo sapiens
gucuuuuuucugugauagagauucuuugcuuuguuagaaacaaaaagcaaagaaucucuaucacagaaaaaagau

SEQ ID NO: 5 - RNA, 88 nt, Homo sapiens
ucccaugcugugacccucuagaggaagcacuuucuguuuguugucugagaaaaaacaaagugcuucccuuuagaguuacuguuuggga

SEQ ID NO: 6 - RNA, 88 nt, Homo sapiens
guauuagguuggugcaaagguaauugcaguuuuucccauuuaaaauauggaaaaaaaaaucacaauuacuuuugcaucaaccuaauaa

SEQ ID NO: 7 - RNA, 22 nt, Homo sapiens
ugagguaguagguuguaugguu

SEQ ID NO: 8 - RNA, 22 nt, Homo sapiens
ugagguaguagauuguauaguu

SEQ ID NO: 9 - RNA, 22 nt, Homo sapiens
ugagguaguaguuuguacaguu

SEQ ID NO: 10 - RNA, 22 nt, Homo sapiens
ugagguaguaguuugugcuguu

SEQ ID NO: 11 - RNA, 22 nt, Homo sapiens
ccgcacuguggguacuugcugc

SEQ ID NO: 12 - RNA, 23 nt, Homo sapiens
uacccuguagaaccgaauuugug

SEQ ID NO: 13 - RNA, 22 nt, Homo sapiens
uuuccggcucgcgugggugugu

SEQ ID NO: 14 - RNA, 22 nt, Homo sapiens
acaggugagguucuugggagcc

SEQ ID NO: 15 - RNA, 22 nt, homo sapiens
acggguuaggcucuugggagcu

SEQ ID NO: 16 - RNA, 22 nt, homo sapiens
ucacaagucaggcucuugggac

SEQ ID NO: 17 - RNA, 18 nt, homo sapiens
aucccaccucugccacca

SEQ ID NO: 18 - RNA, 22 nt, homo sapiens
augguacccuggcauacugagu

SEQ ID NO: 19 - RNA, 22 nt, homo sapiens
ucggauccgucugagcuuggcu

SEQ ID NO: 20 - RNA, 26 nt, homo sapiens
gaugaugauggcagcaaauucugaaa

SEQ ID NO: 21 - RNA, 17 nt, homo sapiens
ucgccuccuccucuccc

SEQ ID NO: 22 - RNA, 22 nt, homo sapiens
uuagggcccuggcuccaucucc

SEQ ID NO: 23 - RNA, 24 nt, homo sapiens
uugcagcugccugggagugacuuc

SEQ ID NO: 24 - RNA, 22 nt, homo sapiens
cagugcaaugaugaaagggcau

SEQ ID NO: 25 - RNA, 22 nt, homo sapiens
uaacagucuacagccauggucg

SEQ ID NO: 26 - RNA, 22 nt, homo sapiens
ugugacugguugaccagagggg

SEQ ID NO: 27 - RNA, 22 nt, homo sapiens
uaacacugucugguaaagaugg

SEQ ID NO: 28 - RNA, 23 nt, homo sapiens
guccaguuuucccaggaaucccu

SEQ ID NO: 29 - RNA, 22 nt, homo sapiens
ugagaacugaauuccauggguu

SEQ ID NO: 30 - RNA, 22 nt, homo sapiens
ugagaacugaauuccauaggcu

SEQ ID NO: 31 - RNA, 22 nt, homo sapiens
ucagugcacuacagaacuuugu

SEQ ID NO: 32 - RNA, 22 nt, homo sapiens
ucucccaacccuuguaccagug

SEQ ID NO: 33 - RNA, 21 nt, homo sapiens
cuagacugaagcuccuugagg

SEQ ID NO: 34 - RNA, 23 nt, homo sapiens
uuaaugcuaaucgugauaggggu

SEQ ID NO: 35 - RNA, 22 nt, homo sapiens
uagcagcacauaaugguuugug

SEQ ID NO: 36 - RNA, 22 nt, homo sapiens
acugcagugaaggcacuuguag

SEQ ID NO: 37 - RNA, 22 nt, homo sapiens
accacugaccguugacuguacc

SEQ ID NO: 38 - RNA, 23 nt, homo sapiens
aacauucauugcugucggugggu

SEQ ID NO: 39 - RNA, 24 nt, homo sapiens
uuuggcaaugguagaacucacacu

SEQ ID NO: 40 - RNA, 23 nt, homo sapiens
uaaggugcaucuagugcagauag

SEQ ID NO: 41 - RNA, 22 nt, homo sapiens
aacuggcccucaaagucccgcu

SEQ ID NO: 42 - RNA, 22 nt, homo sapiens
ccaauauuggcugugcugcucc

SEQ ID NO: 43 - RNA, 22 nt, homo sapiens
uucaccaccuucuccacccagc

SEQ ID NO: 44 - RNA, 23 nt, homo sapiens
cccaguguucagacuaccuguuc

SEQ ID NO: 45 - RNA, 20 nt, homo sapiens
agagguauagggcaugggaa

SEQ ID NO: 46 - RNA, 22 nt, homo sapiens
uagcuuaucagacugauguuga

SEQ ID NO: 47 - RNA, 22 nt, homo sapiens
cauugcacuugucucggucuga

SEQ ID NO: 48 - RNA, 19 nt, homo sapiens
ggggccuggcggugggcgg

SEQ ID NO: 49 - RNA, 22 nt, homo sapiens
cugguuucacaugguggcuuag

SEQ ID NO: 50 - RNA, 22 nt, homo sapiens
uagcaccauuugaaaucgguua

SEQ ID NO: 51 - RNA, 22 nt, homo sapiens
cuuucagucggauguuugcagc

SEQ ID NO: 52 - RNA, 22 nt, homo sapiens
uguaaacauccuacacucagcu

SEQ ID NO: 53 - RNA, 22 nt, homo sapiens
uguaaacauccccgacuggaag

SEQ ID NO: 54 - RNA, 18 nt, homo sapiens
aaagcuggguugagaagg

SEQ ID NO: 55 - RNA, 20 nt, homo sapiens
acugccccaggugcugcugg

SEQ ID NO: 56 - RNA, 21 nt, homo sapiens
gccccugggccuauccuagaa

SEQ ID NO: 57 - RNA, 21 nt, homo sapiens
gaacggcuucauacaggaguu

SEQ ID NO: 58 - RNA, 22 nt, homo sapiens
uccagcaucagugauuuuguug

SEQ ID NO: 59 - RNA, 23 nt, homo sapiens
ugagcgccucgacgacagagccg

SEQ ID NO: 60 - RNA, 23 nt, homo sapiens
ucccuguccuccaggagcucacg

SEQ ID NO: 61 - RNA, 21 nt, homo sapiens
aggggugcuaucugugauuga

SEQ ID NO: 62 - RNA, 22 nt, homo sapiens
gcugacuccuaguccagggcuc

SEQ ID NO: 63 - RNA, 24 nt, homo sapiens
caaagugaugaguaauacuggcug

SEQ ID NO: 64 - RNA, 21 nt, homo sapiens
ucucucggcuccucgcggcuc

SEQ ID NO: 65 - RNA, 24 nt, homo sapiens
aauccuuggaaccuaggugugagu

SEQ ID NO: 66 - RNA, 22 nt, homo sapiens
cggguggaucacgaugcaauuu

SEQ ID NO: 67 - RNA, 22 nt, homo sapiens
aauugcacgguauccaucugua

SEQ ID NO: 68 - RNA, 24 nt, homo sapiens
cauagcccggucgcugguacauga

SEQ ID NO: 69 - RNA, 23 nt, homo sapiens
ugaggauauggcagggaagggga

SEQ ID NO: 70 - RNA, 24 nt, homo sapiens
cccggacaggcguucgugcgacgu

SEQ ID NO: 71 - RNA, 22 nt, homo sapiens
uuuguucguucggcucgcguga

SEQ ID NO: 72 - RNA, 22 nt, homo sapiens
cuccugacuccagguccugugu

SEQ ID NO: 73 - RNA, 21 nt, homo sapiens
acuggacuaggagucagaagg

SEQ ID NO: 74 - RNA, 21 nt, homo sapiens
ugguagacuauggaacguagg

SEQ ID NO: 75 - RNA, 22 nt, homo sapiens
gaaguuguucgugguggauucg

SEQ ID NO: 76 - RNA, 20 nt, homo sapiens
guggguuggggcgggcucug

SEQ ID NO: 77 - RNA, 22 nt, homo sapiens
gaauguugcucggugaaccccu

SEQ ID NO: 78 - RNA, 23 nt, homo sapiens
agcucggucugaggccccucagu

SEQ ID NO: 79 - RNA, 21 nt, homo sapiens
caaaacgugaggcgcugcuau

SEQ ID NO: 80 - RNA, 23 nt, homo sapiens
aaugacacgaucacucccguuga

SEQ ID NO: 81 - RNA, 20 nt, homo sapiens
ucagggagucaggggagggc

SEQ ID NO: 82 - RNA, 18 nt, homo sapiens
gggcucacaucaccccau

SEQ ID NO: 83 - RNA, 17 nt, homo sapiens
accccacuccugguacc

SEQ ID NO: 84 - RNA, 23 nt, homo sapiens
ucuuggaguaggucauugggugg

SEQ ID NO: 85 - RNA, 18 nt, homo sapiens
ggugggcuucccggaggg

SEQ ID NO: 86 - RNA, 22 nt, homo sapiens
cgucccggggcugcgcgaggca

SEQ ID NO: 87 - RNA, 20 nt, homo sapiens
uaacggccgcgguacccuaa

SEQ ID NO: 88 - RNA, 17 nt, homo sapiens
gggagaagggucggggc

SEQ ID NO: 89 - RNA, 23 nt, homo sapiens
agggaaaaaaaaaaggauuuguc

SEQ ID NO: 90 - RNA, 22 nt, homo sapiens
uaggggcagcagaggaccuggg

SEQ ID NO: 91 - RNA, 22 nt, homo sapiens
agcaaggcggcaucucucugau

SEQ ID NO: 92 - RNA, 23 nt, homo sapiens
ugaguggggcucccgggacggcg

SEQ ID NO: 93 - RNA, 24 nt, homo sapiens
aggcaggggcuggugcugggcggg

SEQ ID NO: 94 - RNA, 22 nt, homo sapiens
guggaccaggauggcaagggcu

SEQ ID NO: 95 - RNA, 22 nt, homo sapiens
gcggggguggcggcggcauccc

SEQ ID NO: 96 - RNA, 22 nt, homo sapiens
aguggggaacccuuccaugagg

SEQ ID NO: 97 - RNA, 22 nt, homo sapiens
ugaaacauacacgggaaaccuc

SEQ ID NO: 98 - RNA, 22 nt, homo sapiens
augcaccugggcaaggauucug

SEQ ID NO: 99 - RNA, 23 nt, homo sapiens
uaauccuugcuaccugggugaga

SEQ ID NO: 100 - RNA, 22 nt, homo sapiens
aaugcacccgggcaaggauucu

SEQ ID NO: 101 - RNA, 22 nt, homo sapiens
aaugcaccugggcaaggauuca

SEQ ID NO: 102 - RNA, 22 nt, homo sapiens
ccucccacacccaaggcuugca

SEQ ID NO: 103 - RNA, 22 nt, homo sapiens
caugccuugaguguaggaccgu

SEQ ID NO: 104 - RNA, 25 nt, homo sapiens
aaaaaccacaauuacuuuugcacca

SEQ ID NO: 105 - RNA, 23 nt, homo sapiens
ugagugugugugugugagugugu

SEQ ID NO: 106 - RNA, 21 nt, homo sapiens
uggguuuacguugggagaacu

SEQ ID NO: 107 - RNA, 25 nt, homo sapiens
agggaucgcgggcggguggcggccu

SEQ ID NO: 108 - RNA, 22 nt, homo sapiens
uacccauugcauaucggaguug

SEQ ID NO: 109 - RNA, 24 nt, homo sapiens
acuggcuagggaaaaugauuggau

SEQ ID NO: 110 - RNA, 23 nt, homo sapiens
aaggagcuuacaaucuagcuggg

SEQ ID NO: 111 - RNA, 22 nt, homo sapiens
cugcccuggcccgagggaccga

SEQ ID NO: 112 - RNA, 22 nt, homo sapiens
acugcugagcuagcacuucccg

SEQ ID NO: 113 - RNA, 22 nt, homo sapiens
caagcucgugucuguggguccg

SEQ ID NO: 114 - DNA, 74 bp, homo sapiens
tggaccaatgatgagtaccatggggtatctgaaacaggatttttgattaaacccatatgcaattctgaggtcca

SEQ ID NO: 115 - DNA, 74 bp, homo sapiens
tggaccaatgatgacaactgccggcgtatgagtgttgggtgatgaataatacgtgtctagaactctgaggtcca

SEQ ID NO: 116 - DNA, 74 bp, homo sapiens
tggaccaatgatgaccactggtggcgtttgagtcatggacgatgaatactacgtgtctgaaactctgaggtcca

SEQ ID NO: 117 - DNA, 97 bp, homo sapiens
ccggggcctccatgatgtccagcactgggctccgactgccactgaggacacggtgccccccgggacctttgacacccgggggtctgaggggccctgg

SEQ ID NO: 118 - DNA, 97 bp, homo sapiens
ttggggaccccgtgatgtccagcactgggctctgactgcccctgaggacacggtgcaccccgggacctttgacatccggggttctgaggggccccac

SEQ ID NO: 119 - DNA, 97 bp, homo sapiens
ctggggctcccatgatgtccagcactgggctctgatcacccctgaggacacagtgcaccccaggacctttgacacctgggggtctgaggggccccag

SEQ ID NO: 120 - DNA, 77 bp, homo sapiens
aatgtgaagcaaatgatgataaactggatctgactgactgtgctgagtctgttcaatccaaccctgagcttcatgtt

SEQ ID NO: 121 - DNA, 76 bp, homo sapiens
acaatgatgacttaaattactttttgccgtttacccagctgaggttgtctttgaagaaataattttaagactgaga

SEQ ID NO: 122 - DNA, 114 bp, homo sapiens
actgaggaatgatgacaagaaaaggccgaattgcagtgtctccatcagcagtttgctctccatgggcacacgatgacaaaatatcctgaagcgaaccactagtctgacctcagt

SEQ ID NO: 123 - DNA, 89 bp, homo sapiens
tggtgctgtgatgatgccttaatattgtggtttcgactcactgagagtaaaatgaggacctacaattccttggctgtgtctgagcaccc

SEQ ID NO: 124 - DNA, 75 bp, homo sapiens
ttgcagtgatgacttgcgaatcaaatctgtcaatcccctgagtgcaatcactgatgtctccatgtctctgagcaa

SEQ ID NO: 125 - DNA, 98 bp, homo sapiens
tggatcgatgatgactataaaaaaaatggatctcatcggaatctgaacaaaatgagtgaccaaatcatttctgtgccacttctgtgagctgaggtcca

SEQ ID NO: 126 - DNA, 98 bp, homo sapiens
tggatcgatgatgagtcctccaaaaaaaacattccttggaaaagctgaacaaaatgagtgaaaactcataccgtcattctcatcggaactgaggtcca

SEQ ID NO: 127 - DNA, 70 bp, homo sapiens
ggtgaaaatgatgaattctggggcgctgattcatgtgacttgaaaaatgccatccatttcctgattcacc

SEQ ID NO: 128 - DNA, 79 bp, homo sapiens
ccacatgcggctgatgacagcacttctgctgagacgctgtgattgctctgtccaaagtaaacgccctgacgcactgtgg

SEQ ID NO: 129 - DNA, 148 bp, homo sapiens
cttcgatgaagagatgatgacgagtctgacttggggatgttctctttgcccaggtggcctactctgtgctgcgttctgtggcacagtttaaagagccctggttgaagtaatttcctaaagatgacttagaggcatttgtctgagaagg

SEQ ID NO: 130 - DNA, 146 bp, homo sapiens
cttcagtgatgacacgatgacgagtcagaaaggtcacgtcctgctcttggtccttgtcagtgccatgttctgtggtgctgtgcacgagttcctttggcagaagtgtcctatttattgatcgatttagaggcatttgtctgagaagg

SEQ ID NO: 131 - DNA, 95 bp, homo sapiens
gctgaatgatgatatcccactaactgagcagtcagtagttggtcctttggttgcatatgatgcgataattgtttcaagacgggactgatggcagc

SEQ ID NO: 132 - DNA, 67 bp, homo sapiens
ttcctatgatgaggaccttttcacagacctgtactgagctccgtgaggataaataactctgaggaga

SEQ ID NO: 133 - DNA, 72 bp, homo sapiens
actccatgatgaacacaaaatgacaagcatatggctgaactttcaagtgatgtcatcttactactgagaagt

SEQ ID NO: 134 - DNA, 75 bp, homo sapiens
gtcagatgatttgaattgataagctgatgttctgtgaggtacaaaagttaatagcatgttagagttctgatggca

SEQ ID NO: 135 - DNA, 65 bp, homo sapiens
tttctatgatgaatcaaactagctcactatgaccgacagtgaaaatacatgaacacctgagaaac

SEQ ID NO: 136 - DNA, 217 bp, homo sapiens
aagactatactttcagggatcatttctatagtgtgttactagagaagtttctctgaacgtgtagagcaccgaaaaccccgaggaagagaggtagcgttttctcctgagcgtgaagccggctttctggcgttgcttggctgcaactgccgtcagccattgatgatcgttcttctctccgtattggggagtgagagggagagaacgcggtctgagtggt

SEQ ID NO: 137 - DNA, 217 bp, homo sapiens; identical to SEQ ID NO: 136

SEQ ID NO: 138 - DNA, 217 bp, homo sapiens
aaggctatactttcagggatcatttctatagtgtgttactagagaagtttctttgaacgtgtagagcaccgaaaaccccgaggaagagaggtagcgttttctcctgagcgtgaagccggctttctggcgttgcttggctgcaactgccgtcagccattgatgatcgttcttctctccgtattggggagtgagagggagagaacgcggtctgagtggt

SEQ ID NO: 139 - DNA, 70 bp, homo sapiens
gtttgtgatgacttacatggaatctcgttcggctgatgacttgctgttgagactctgaaatctgattttc

SEQ ID NO: 140 - DNA, 71 bp, homo sapiens
ctcaccagtgatgagttgaataccgccccagtctgatcaatgtgtgactgaaaggtattttctgagctgtg

SEQ ID NO: 141 - DNA, 77 bp, homo sapiens
gtcagtgatgagcaacattcaccatctttcgtttgagtctcacggccatgagatcaaccccatgcaccgctctgaga

SEQ ID NO: 142 - DNA, 83 bp, homo sapiens
ggccggtgatgagaacttctcccactcacattcgagtttcccgaccatgagatgactccacatgcactaccatctgaggccac

SEQ ID NO: 143 - DNA, 86 bp, homo sapiens
ggcagatgatgtccttatctcacgatggtctgcggatgtccctgtgggaatggcgacaatgccaatggcttagctgatgccaggag

SEQ ID NO: 144 - DNA, 71 bp, homo sapiens
gttgcagtgatgtaaaatttcttggcctgaaattactgtgaagagtaaaaccgagctttttaacactgagt

SEQ ID NO: 145 - DNA, 71 bp, homo sapiens
ttctcgtgatgaaaactctgtccagttctgctactgaagggagagagatgagagccttttaggctgaggaa

SEQ ID NO: 146 - DNA, 69 bp, homo sapiens
tctcagtgatgaaaactttgtccagttctgctactgacagtaagtgaagataaagtgtgtctgaggaga

SEQ ID NO: 147 - DNA, 70 bp, homo sapiens
tgggaagtgatgacacctgtgactgttgatgtggaactgatttatcgcgtattcgtactggctgatcctg

SEQ ID NO: 148 - DNA, 58 bp, homo sapiens
aatgatggaaaaatcattattggaaaagaatgacatgaacaaaggaaccactgaagtg

SEQ ID NO: 149 - DNA, 63 bp, homo sapiens
cctggatgatgataagcaaatgctgactgaacatgaaggtcttaattagctctaactgactaa

SEQ ID NO: 150 - DNA, 98 bp, homo sapiens
gtagggtgatgaaaaagaatccttaggcgtggttgtggccgtcttggtcacctgtgtgccacttgccaatgcaaggacttgtcatagttacactgact

SEQ ID NO: 151 - DNA, 64 bp, homo sapiens
agtgatgatgaccccaggtaactcttgagtgtgtcgctgatgccatcaccgcagcgctctgacc

SEQ ID NO: 152 - DNA, 71 bp, homo sapiens
tgctctgatgaaatcactaataggaagtgccgtcagaagcgataactgacgaagactactcctgtctgatt

SEQ ID NO: 153 - DNA, 71 bp, homo sapiens
ccacaatgatggcaatatttttcgtcaacagcagttcacctagtgagtgttgagactctgggtctgagtga

SEQ ID NO: 154 - DNA, 75 bp, homo sapiens
ccttctatgatgattttatcaaaatgactttcgttcttctgagtttgctgaagccacatttaggtactgagaagg

SEQ ID NO: 155 - DNA, 75 bp, homo sapiens
tattcctcactgatgagtacgttctgactttcgttcttctgagtttgctgaagccagatgcaatttctgagaagg

SEQ ID NO: 156 - DNA, 65 bp, homo sapiens
aataagtgatgaaaaaagtttcggtcccagatgatggccagtgataacaacatttttctgatgtt

SEQ ID NO: 157 - DNA, 72 bp, homo sapiens
ctgcctctgatgaagcctgtgttggtagggacatctgagagtaatgatgaatgccaaccgctctgatggtgg

SEQ ID NO: 158 - DNA, 60 bp, homo sapiens
agcctgtgatgctttaagagtagtggacagaagggatttctgaaattctattctgaggct

SEQ ID NO: 159 - DNA, 80 bp, homo sapiens
gccacaatgatgacagtttatttgctactcttgagtgctagaatgatgaggatcttaaccaccattatcttaactgaggc

SEQ ID NO: 160 - DNA, 65 bp, homo sapiens
gtgtaatgatgttgatcaaatgtctgacctgaaatgagcatgtagacaaaggtaacactgaagaa

SEQ ID NO: 161 - DNA, 95 bp, homo sapiens
gctgttcgttgatgaggctcagagtgagcgctgggtacagcgcccgaatcggacagtgtagaaccattctctactgccttccttctgagaacagc

SEQ ID NO: 162 - DNA, 72 bp, homo sapiens
cctggtgatgacagatggcattgtcagccaatccccaagtgggagtgaggacatgtcctgcaattctgaagg

SEQ ID NO: 163 - DNA, 72 bp, homo sapiens
ggtgcagatgatgacactgtaaagcgaccaaagtctgaacaaagtgattggtacctcgttgtctgatgcacc

SEQ ID NO: 164 - DNA, 71 bp, homo sapiens
gatgttatgatgatgggcgaaatgttcaactgctctgaaggggctgaatgaaaatggcctttctgaacatc

SEQ ID NO: 165 - DNA, 70 bp, homo sapiens
aagtgaaatgatggcaatcatctttcgggactgacctgaaatgaagagaatactcattgctgatcacttg

SEQ ID NO: 166 - DNA, 133 bp, homo sapiens
ggtctctcagctccgcttaaccacacgggtccagtgtgtgcttggcgtgttttcagggaggcagagaaaggctctcctaatgcacgacagacccgcccagaatggcctctctgttcctaggagtgcgacaatt

SEQ ID NO: 167 - DNA, 132 bp, homo sapiens
gttgaggtctatcccgatggggcttttcctgtagcctgcacatcgttggaaacgcctcatagagtaactctgtggttttactttactcacaggactattgttagatctgtgggaaggaattacaagacagtt

SEQ ID NO: 168 - DNA, 132 bp, homo sapiens
cttcccatttatttgctgcttgtagtctcacagtgatacgagcagttatacgcatgggataaaataacattgggccactgtaaattgagatgaagtaaccattttcatctcttctgcagggactagacattg

SEQ ID NO: 169 - DNA, 133 bp, homo sapiens
cccccttttaaaagcactcaatgggcctgtggctaatgacctattgagccgtcaagaaaggggagagtgaaaacatcgcttttgggtgaagtggcaacatgtgttgtttgcttcaatcggtggtgtgacaagg

SEQ ID NO: 170 - DNA, 133 bp, homo sapiens
aagccagccaatgaatctgcttacctgattgtgtttgtgcagacatactttaaaaactggcaatagtaaagccatgttacgagccttaaggacattgaagtcgttaaggtccctgagaatggctataacaaat

SEQ ID NO: 171 - DNA, 137 bp, homo sapiens
gtggccctgactgaagaccagcagttgtactgtggctgttggtttcaagcagaggcctaaaggactgtcttcctgtggtctgttggctgttctgggacctcagtagggaatggctatttcatttggaagaaacaacc

SEQ ID NO: 172 - DNA, 130 bp, homo sapiens
atcgaggctagagtcacgcttgggtatcggctattgcctgagtgtgctagagtcctcgaagagtaactgctgaccttattcactggctgtgggccttatggcacagtcagtcaccaggttagagacatgc

SEQ ID NO: 173 - DNA, 134 bp, homo sapiens
tggtaatggatttatggtgggtccttctctgtgggcctctcatagtgtacccatgccatagcaaatggcagcctcgaaccattgcccagtccccttacctgtgggctgtgagcactgaagggggttgcacagtg

SEQ ID NO: 174 - DNA, 132 bp, homo sapiens
cagcatgtttccaagggctgtggctggtcatagccatgggatctccaactgcatgcaagagcaacctggaaagactttgacagcgcaggtcagtacaatacctgcaagctgccactcagctttcctataatg

SEQ ID NO: 175 - DNA, 135 bp, homo sapiens
tgtccctgacctgggtagagtggcatctggttggtgatgcccatctcatatcagccagggacaaagcaactccttgttcatcccagcttggcttttgatccgtgcccatgcctggttcatgccttggacacatag

SEQ ID NO: 176 - DNA, 134 bp, homo sapiens
tggtccatcctaatccctgccggtccatctgtggcctgccaggtttcgcttgtggaccagagcaccctagaagcctcacccgaggagtgagcagggctccagtgggctcacgtcatgggcacttctagacactc

SEQ ID NO: 177 - DNA, 123 bp, homo sapiens
gagcactgttcgtaacccgttagcctggctgtagctaatgggttccattccggtgcaatagcatttccagcgacacatgactgactgactggtggctttcagtttcaggtcttggagacaaat

SEQ ID NO: 178 - DNA, 137 bp, homo sapiens
gagcacctgaatctttcccattccttgctgcctcgtgccggtgtggggacagatggtgctacagaatgagcagaggaaatccagacaggttgttttccatttgtcttggggcctgtctctacagctctgccacattt

SEQ ID NO: 179 - DNA, 133 bp, homo sapiens
gcgctgtctttgagcccccgccgagcttcctcgtggcgccgggggtcaatctgcagcgctagagcatgtgcttgcgcataactggggccgcctggcctcccgcgggcggcctttttaaccgcgagcgacaaga

SEQ ID NO: 180 - DNA, 149 bp, homo sapiens
tgcacactattaaagctcagggtggaggccagtcttggctcatgaacttctgagtgtcggaagtgtgctatatcaatggcaggattttcgctaacaccagtagagcttgcctctatgactggagtttggtagtactcgctgccacatag

SEQ ID NO: 181 - DNA, 133 bp, homo sapiens
tagcaagcctccagcgtgcttgggtctgcggtgaccctatgcattccttcagtgcttgctagaacagttttgaaacggtttgaggccttgccctgctccatccagagcaaggttatagaaatttcagacaatg

SEQ ID NO: 182 - DNA, 207 bp, homo sapiens
tccaacgtggatacacccgggaggtcactctccccgggctctgtccaagtggcgtaggggagcatagggctctgccccatgatgtacaagtccctttccacaacgttggaaataaagctgggcctcgtgtctgcgcctgcatattcctacagcttcccagagtcctgtcgacaattactggggagacaaaccatgcaggaaacagcc

SEQ ID NO: 183 - DNA, 205 bp, homo sapiens
tccaacgtggataccctgggaggtcactctccccaggctctgtccaagtggcataggggagcttagggctctgccccatgatgtacagtccctttccacaacgttgaagatgaagctgggcctcgtgtctgcgcctgcatattcctacagcttcccagagtcctgtggacaatgactggggagacaaaccatgcaggaaacatat

SEQ ID NO: 184 - DNA, 200 bp, homo sapiens
atccagcggttgtcagctatccaggctcatgtggtgcctgtgatggtgttacactgttggaagagcaaacactgtctttattgaggtttggctccaagcactgttttggtgttgtagctgagtacctttgggcagtgttttgcacctctgagagtggaatgactcctgtggagttgatcctagtctgggtgcaaacaatt

SEQ ID NO: 185 - DNA, 137 bp, homo sapiens
gtcttctcattgagctcctttctgtctatcagtggcagtttatggattcgcacgagaagaagagagaattcacagaactagcattattttaccttctgtctttacagaggtatatttagctgtattgtgagacattc

SEQ ID NO: 186 - DNA, 134 bp, homo sapiens
actctctcggctctgcatagttgcacttggcttcacccgtgtgactttcgtaacggggagagagagaaaagatctcctcaggacctcggatgggccttactgtggcctctctttccttgaggggtgcaacaggc

SEQ ID NO: 187 - DNA, 133 bp, homo sapiens
gtgcaaactcgatcactagctctgcgtgatgtggcagaagcgaagggaaccaggtttgcaaaagtaactgtggtgatggaaatgtgttagcctcagacactactgaggtggttctttctatcctagtacagtc

SEQ ID NO: 188 - DNA, 133 bp, homo sapiens
attgcacctaaacccaagaatcactgtttcttatagcggtggtttaaacagaggtgcaaacagcaagcggatcttgtcgcctttggggggctgtggccgtgcccctcaaagtgaatttggaggttccacaact

SEQ ID NO: 189 - DNA, 138 bp, homo sapiens
cacctgtattcgaaagtgatcgtgggctgcctgtgccctggtcattgatagtgcagggaaagaaatcgcggaaagtgcttccccgtgtttggagggtccgctcctgtccctttcaaactctggagctttctcacacct

SEQ ID NO: 190 - DNA, 421 bp, homo sapiens
agaggcttgggccgccgagctggacccggaccggttttgggtactgtactgggggcagggcagagaggtgggcggcagttggggtgcggtgattgtagtaggctagggcgctttcgggtccccattgcagcccccggatgagcccgcagtattttccttatatgatcaggtcccattgcgggcggcgccgcttgcccggagcctgagaggattatgaaaacgtggcgagcgaaatggggccaggggacctggagcaggggcgtgaggagagtaggcagcgggtgaggctggacgggagggaggtctagggaggcctctgccgcgggcactgtgagtcctggccgatgatgacgagaccactgcgcaatctgagttctgggaaccaggtgatggagtatgttctgagaacagactgaggccg

SEQ ID NO: 191 - DNA, 104 bp, homo sapiens
tgctggagtgatgaaaaagtatcttcaggtgtggctgtggccaccttggccacctgtgtgtcacttgccaatgcaaggacttgtcatagttacactgactgtta

SEQ ID NO: 192 - DNA, 131 bp, homo sapiens
ccctcctacaaaggcatgtctataattccttgtctttggacatgtaagaattggagggacagaaatgtggacttggagaaatctggggccagctttctcatcacaggctcaacatcaaccatgccacatag

SEQ ID NO: 193 - DNA, 92 bp, homo sapiens
agatcattgatgacttccatatatccattccttggaaagctgaacaacatgagtgaaaactctactgaaaaaagaaaagaaatgggaggccg

SEQ ID NO: 194 - DNA, 131 bp, homo sapiens
ctccatgtatctttgggacctgtcaagtgtggcagtctcccttccttgccatggaagagcatattcttgtttaccagcaaagctgtcaccatttaattggtatcagattctgacttgcacaagtaacattc

SEQ ID NO: 195 - DNA, 139 bp, homo sapiens
gagcttccaggatcacccctgcagagtggctaatattctgccagcttcggaaagggaggggaagcaagcctggcagaggcacccattccattcccagcttgcttagtagctggccatgggaagacactgtgcaacactg

SEQ ID NO: 196 - DNA, 133 bp, homo sapiens
ggtctctcagctctgcttaaccacacgggtccagtgtgtgcttggcgtgttttcagggaggcagagaaaggctctcctaatgcacgacagacccgcccagaatggcctctctgttcctaggagtgtgacaatt

SEQ ID NO: 197 - DNA, 69 bp, homo sapiens
atgtaataatgttcatcaaatgtctgacctgaaatgagcatgtagacaagttaatttaacactgaagaa

SEQ ID NO: 198 - DNA, 128 bp, homo sapiens
tgcacttatgtatgtttttgtttaacttgtggacaaagactttaggaaaggtgcaaaaaataaatcttcttttgcaacccagaactcattgttcagtatgagttttgatacatatcagaatggatact

SEQ ID NO: 199 - DNA, 129 bp, homo sapiens
agggtttgcttagggcagggaggttgaagagtggctcctctgtttacaatacaccaaacaggaatctggggtcattgtgacaaggggcacaaaacttgtgtcctccctacatgtgaaaaaaaaaaaaga

SEQ ID NO: 200 - DNA, 197 bp, homo sapiens
aagactgtactttcaggaatcatttctatagttcattactagagaaatttctctgaacatgtagagcaccagaaaatatttttaaagatttctttaggctgggcgtggtggctcacgcctgtaatcccagcactttgggaggccgaagtgggcggatcatctgaggtcgggagttcgagaccagcctgactaacatg

SEQ ID NO: 201 - DNA, 104 bp, homo sapiens
atccttttgtagttcttaagtgtgatgattgggttttcatgcttatgtgtgaaatgtgcctttctcaaaccttgttatgacactggcacattacctgtgtgacg

SEQ ID NO: 202 - DNA, 100 bp, homo sapiens
aaattttaggagtacctaagtgtgatgatttggttttcacattcatgtgtgagctgtgcctgccttttgttacaagggcatattaccctttgttgtgaaa

SEQ ID NO: 203 - DNA, 34 bp, homo sapiens
actgacctgaaatgaagagaatactcattgctga

SEQ ID NO: 204 - DNA, 104 bp, homo sapiens
gtccacttgtagttcataagcaacatgatttggttttcatgctgatgtgtgagatgtgcctccctcaaaccttgttactatgttggcacattacaagtttgaca

SEQ ID NO: 205 - DNA, 104 bp, homo sapiens
atcctttcgtagtttataagagtgatgattaggtcttcatgctcatgtgtgaaatgtgcctccctcaaaccatgttaggacgttggcatattgcccatctgaaa

SEQ ID NO: 206 - DNA, 104 bp, homo sapiens
atccttttgtagtttatgagcatgatgactgggttttcacaggtatgtgtgagatgtgccatcctcgaaccttgttatgatgtcggcatattgtcagtctgaca

SEQ ID NO: 207 - DNA, 96 bp, homo sapiens
cttctgctaaggtttacactatagatgcaggaaaaaaaatgtcctcacactgtctgtctgattgtggcagctgagattgaatagagaaatataggg

SEQ ID NO: 208 - DNA, 85 bp, homo sapiens
ggattgacgatgactttaaaaaaaaaaaatctcattgaaatctgaaaaaaatgagtgaccaaaccacttctgtgagctgaggtcc

SEQ ID NO: 209 - DNA, 141 bp, homo sapiens
aagactatactttcagggatcatttctatagttctttactagagaagtttctctgaacatgtagagcactgtgccttaaaaaagaaaaaaaaaagggctgggcatggtggctcacgcctgtaatcccagcactttgggagg

(19) United States
(12) Patent Application Publication
(10) Pub. No.: US 2019/0144951 A1
(43) Pub. Date: May 16, 2019

(54) METHODS AND COMPOSITIONS FOR PROSTATE CANCER DIAGNOSIS AND TREATMENT

(71) Applicants: MIR DIAGNOSTICS, Rensselaer, NY (US); The Research Foundation for The State University of New York, Albany, NY (US)

(72) Inventors: Martin TENNISWOOD, Rensselaer, NY (US); Wei-Lin Winnie WANG, Rensselaer, NY (US); Gregory DIRIENZO, Rensselaer, NY (US); Tucker CONKLIN, Rensselaer, NY (US)

(73) Assignees: miR Scientific, LLC, Rensselaer, NY (US); The Research Foundation for The State University of New York, Albany, NY (US)

(21) Appl. No.: 16/307,526

(22) PCT Filed: Jun. 8, 2017
(86) PCT No.: PCT/US2017/036611

§ 371 Date: Dec. 6, 2018

Related U.S. Application Data
(60) Provisional application No. 62/347,600, filed on Jun. 8, 2016.

(51) Int. Cl. C12Q 1/6886; G16H 50/30; G16H 50/20; C12Q 1/6825

(52) U.S. Cl. CPC C12Q 1/6886; G16H 50/30; G16H 50/20; C12Q 2600/158; C12Q 2600/106; C12Q 2600/112; C12Q 2600/118; C12Q 1/6825

(57) ABSTRACT

The present disclosure relates to compositions and methods for diagnosing, prognosing, monitoring, and treating a patient with prostate cancer. In particular, the disclosure relates to ncRNAs as diagnostic markers for determination of proper treatment administration.

### CROSS-REFERENCE TO RELATED APPLICATIONS

This application claims the benefit of U.S. provisional applications 62/347,600 filed Jun. 8, 2016, which is incorporated herein by reference in its entirety.

### FIELD OF THE INVENTION

The present disclosure relates to compositions and methods for diagnosing, prognosing, monitoring, and treating a patient with prostate cancer. In particular, the disclosure relates to ncRNAs and miRNAs as diagnostic markers for determination of proper treatment administration.

### BACKGROUND

In November 2012, the U.S. Preventive Services Task Force recommended against prostate-specific antigen (PSA) screening for prostate cancer in healthy men, because of the “moderate to high certainty that the service has no benefit and that the harms actually outweigh the benefits” (Moyer, 2012). While the task force acknowledges that the test is sensitive and accurate with a low false positive rate, they contend that “there is little evidence PSA testing saves lives but rather that many men instead suffer from impotence, incontinence, heart attacks related to treatment of tiny tumors that would never kill them.” Not surprisingly, many urologists disagree, and a number of prominent urologic oncologists have published strongly dissenting opinions (McNaughton-Collins & Barry, 2011; Catalona et al., 2012). Indeed, a careful, population-based study of the Surveillance Epidemiology and End Results (SEER) data base, using SEER-Stat and Joinpoint regression analyses, that took into account consistent decade followup to determine incidence based mortality and annual percent change, concludes that early detection through PSA screening extends life (Wachtel et al., 2013).

In response to the continuing controversy, the American Urological Association issued new guidelines at its annual meeting in May 2013 (Carter et al., 2013), suggesting that men over the age of 75 should no longer be screened, and men under the age of 45 should be counseled against screening. At the center of this controversy is the fact that only 3 out of 10 men diagnosed with prostate cancer on the basis of PSA testing and a positive needle biopsy need definitive therapy to treat an aggressive (lethal) tumor, while 7 out of 10 men diagnosed the same way have indolent (non-lethal) disease and will never need treatment. Unfortunately, neither Gleason Score nor PSA levels discriminate between indolent and aggressive disease, and in the absence of a test that does so, nearly all men diagnosed with prostate cancer are being treated as though they have aggressive disease, costing the healthcare system an estimated $2.6B per year in unnecessary treatment costs. The significant morbidities associated with radical prostatectomy, including erectile dysfunction, incontinence, inguinal hernia and compromised bowel function are estimated to add 50% to total cost of treatment compared to Watchful Waiting/Active Surveillance (AS) (National Collaborating Centre for Cancer, 2008; Ramsay et al., 2012). It is even more difficult to estimate the very considerable emotional impact of these side effects on patients and their families, and the effect on the Quality-Adjusted Life Years (QALY) measure (Liatsikos et al., 2008; Mirza et al., 2011).

As outlined above, the issues related to over-diagnosis and over-treatment of prostate cancer lies not with the PSA test itself, but with the current inability to distinguish between indolent and aggressive disease once the tumor has been confirmed by needle biopsy. This problem has been recognized for at least 20 years, if not longer (Partin et al., 1992; Coffey, 1993), and remains a major issue today (Keller et al., 2007; Getzenberg & Carter, 2012). In the intervening years, there have been many attempts to develop prognostic markers for aggressive disease, including ploidy, nuclear morphology and nuclear matrix architecture, microarray-based transcriptome analyses, DNA methylation status, and detection of PCA3 and TMPRSS2:ERG gene fusions (Mohler et al., 1992; Partin et al., 1993; Ross et al., 1999; Leman & Getzenberg, 2002; Phé et al., 2010; Salagierski & Schalken, 2012; Bismar et al., 2013). None of these methodologies have proved to be significantly better than Gleason Scores as indicators of prostate tumor progression and they do not adequately identify indolent disease (Velonas et al., 2013). In the absence of better prognostic indicators, there has even been discussion of changing the designation of Gleason 6 adenocarcinoma as “cancer” to discourage immediate and unnecessary intervention (Carter et al., 2012). However the recent reassessment of Gleason grade, taking into account changes in the review criteria (Montironi et al., 2010), lead time bias and other factors, suggests that Gleason Score progression is uncommon, and that the biology of prostate tumor progression is not captured by changes in Gleason Score (Penney et al., 2013). The controversy surrounding the value of Gleason Score in identifying indolent tumors leaves physicians and patients with no reliable measures on which to base their decisions regarding treatment options, resulting in many men needlessly opting for clinical intervention. It also handicaps the development of new prognostic tools since the “gold standard” of the Gleason Score is not itself a reliable indicator of prostate tumor progression.

In the past, tests have been developed that are designed to distinguish indolent and aggressive disease using mRNA expression profiles, however, each demonstrates significant pitfalls and shortcomings. First, with one exception, all of these assays have used tumor material derived from radical prostatectomy specimens, and therefore at best are predictive of early tumor recurrence. While potentially useful for making post-surgical decisions related to continuing clinical decisions, they do not address issues related to distinguishing indolent and aggressive prostate cancer prior to surgery. Secondly, a number of these genomic approaches have focused on specific pathways that have been implicated in prostate cancer progression, including the androgen receptor (AR) modulated gene expression (Heemers et al., 2011), epithelial-stromal interactions (Chen et al., 2012), and cell cycle (Cuzick et al., 2011, 2012; Freeland et al., 2013; Cooperberg et al., 2013). These assays are based on the assumption that all prostate tumor progress along a common pathway. Other commercially available biomarker assays utilize mRNA expression profiles generated by real-time PCR of a small subset of genes.

The association of dysregulated miRNA expression in human solid tumors, including breast and prostate cancer was first described in 2006 (Volinia et al., 2006). Comparison of miRNA expression in the transplantable LuCaP family of prostate xenograft models as well as androgen receptor positive and negative human prostate cancer cell lines was used to define a 30 miRNA signature that distinguished between benign prostatic hyperplasia (BPH) and prostate carcinoma from radical prostatectomies (Porkka et al., 2007). These studies also suggested that a 21 miRNA signature was potentially predictive of the emergence of castration resistant prostate cancer (CRPC). Even though the potential clinical utility of miRNAs has been well recognized (deVere White, 2009; Ha, 2011; Casanova-Salas et al., 2012; Maugeri-Sacca et al., 2012), to date there has been no followup validation studies from this group using clinical material.

Several recent studies have surveyed the expression of selected miRNAs in tumors derived from radical prostatectomies and peritumoral normal tissue (Siva et al., 2009; Carlsson et al., 2011; Schubert et al., 2013), high grade prostate intraepithelial neoplasia and metastatic disease (Leite et al, 2013), or normal epithelial tissue and low (Gleason 6) and high (≥Gleason 8) grade tumors (Walter et al., 2013).

These studies have used a variety of different platforms including commercially available SYBR-Green PCR, TaqMan® PCR, Transcription-Mediated Amplification (TMA) or Deep Sequencing (454 pyro-sequencing) using cryo-preserved or formalin-fixed paraffin-embedded (FFPE) tumor tissue from radical prostatectomies to associate a small number of miRNAs with different stages of prostate cancer progression (Schaefer et al., 2010; Szczyrba et al., 2010). The studies have all focused on developing markers for tumor recurrence after radical prostatectomy, and have not been tested in a prognostic setting.

To date there has been only one genome wide transcriptome study of ncRNAs in prostate cancer (Martens-Uzunova et al., 2012). This research compared the miRNA and snoRNA signatures in freshly frozen radical prostatectomy samples and adjacent normal tissue from the same patient using Illumina/Solexa deep sequencing and microarray analysis on the Affymetrix miRNA V2 microarrays that contains 723 human miRNAs catalogued in Sanger miRBase V10.1. These studies provide a valuable data set for comparing the complement of ncRNAs expressed in prostate cancer and peritumoral benign tissue, but are not useful for the rational design of a panel of ncRNAs that will be prognostic for tumor progression prior to clinical intervention. It is also handicapped as a general screening technology since the technique requires flash frozen material.

Therefore, a test is needed that accurately identifies patients with aggressive prostate cancer, so definitive treatment can be provided quickly for those patients that require it, while also identifying patients with indolent prostate cancer that do not require treatment, thereby avoiding unnecessary expense and compromise of lifespan and health. Further, a test is required that provides clinically actionable information prior to the initiation of any therapeutic intervention such as, for example, surgery or radiation. A need exists for a test that does not interfere with current work-flow in the urology practice or the histopathology laboratory responsible for routine diagnostic evaluation of tumor sections. Finally, a test is needed that can be performed, and results available to the uro-oncology team and the patient in a timely manner so that the results can be used to plan treatment before any treatment is initiated.

### SUMMARY OF THE INVENTION

One aspect of the present disclosure provides a method for diagnosing indolent or aggressive prostate cancer in a subject including a) obtaining a biological sample from a human patient; b) detecting the expression level of at least 10 ncRNAs selected from the group consisting of SEQ ID NOs:1-209 by contacting the biological sample with a reagent in an in vitro assay; and c) identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample.

In some embodiments, the biological sample is selected from the group consisting of prostate tissue and prostate cells. In other embodiments, the tissue is formalin-fixed paraffin-embedded tissue. In certain embodiments, the method includes extracting ncRNA from the biological sample.

In some embodiments, the detecting is selected from the group consisting of reverse transcription polymerase chain reaction, polymerase chain reaction, and nucleic acid hybridization, or any combination thereof. In certain embodiments, the reagent is selected from the group consisting of oligoribonucleotide primers, oligonucleotide primers, oligoribonucleotide probes, and oligonucleotide probes, or any combination thereof.

In some embodiments, the ncRNAs are selected from the group consisting of miRNA, C/D box snoRNA, H/ACA box snoRNA, scaRNAs, piRNAs, and lncRNAs or any combination thereof.

Another aspect of the present disclosure provides a method of screening a subject for indolent or aggressive prostate cancer including a) hybridizing ncRNAs from a biological sample from the subject with a microarray comprising probes for whole-genome ncRNAs; b) detecting the relative abundance of hybridization products for at least 10 ncRNAs selected from the group consisting of SEQ ID NOs:1-209; and c) comparing the cumulative expression levels of the at least 10 ncRNAs from the biological sample with the cumulative expression levels of the at least 10 ncRNAs from an indolent prostate cancer biological sample, wherein an increased level of expression of the at least 10 ncRNAs in the subject is indicative of aggressive prostate cancer in further need of treatment, and wherein an equal or less than level of expression of the at least 10 ncRNAs in the subject is indicative of indolent prostate cancer not in need of further treatment.

In some embodiments, the biological sample is selected from the group consisting of prostate tissue and prostate cells. In other embodiments, the tissue is formalin-fixed paraffin-embedded tissue. In certain embodiments, the method includes extracting ncRNA from the biological sample.

In some embodiments, the detecting is selected from the group consisting of reverse transcription polymerase chain reaction, polymerase chain reaction, and nucleic acid hybridization, or any combination thereof. In certain embodiments, the reagent is selected from the group consisting of oligoribonucleotide primers, oligonucleotide primers, oligoribonucleotide probes, and oligonucleotide probes, or any combination thereof.

Yet another aspect of the present disclosure provides a method of treatment of aggressive prostate cancer in a subject including a) obtaining a biological sample from a human patient; b) detecting the expression level of at least 10 ncRNAs selected from the group consisting of SEQ ID NOs:1-209 by contacting the biological sample with a reagent in an in vitro assay; c) identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample; and d) treating the aggressive prostate cancer.

In some embodiments, the treating is selected from the group consisting of i) surgery for partial or complete surgical removal of prostate tissue; ii) administering an effective dose of radiation; and iii) administering a therapeutically effective amount of a medication for the treatment of aggressive prostate cancer.

In certain embodiments, the surgery is chosen from laparoscopic surgery, laparoscopic radical prostatectomy, prostatectomy, and radical retropubic prostatectomy. In other embodiments, the radiation is chosen from external beam radiotherapy, brachytherapy, and particle beam therapy. In some embodiments, the medication for the treatment of aggressive prostate cancer is chosen from a chemotherapeutic and a sex hormone suppressor. In yet other embodiments, the chemotherapeutic is chosen from docetaxel (Taxotere), cabazitaxel (Jetvana), Goserelin (Zoladex), Flutamide (Eulexin), Bicalutamide (Casodex), Abiraterone (Zytiga), and Nilutamide (Nilandron). In some embodiments, the sex hormone suppressor is chosen from Leuprolide (Lupron).

In some embodiments, the treatment for aggressive prostate cancer can be determined, in whole or in part, by the combined expression level of the at least 10 ncRNAs.

### BRIEF DESCRIPTION OF THE DRAWINGS

FIG. 1 is schematic representation of current clinical practice for diagnosis of prostate cancer in patients.

FIG. 2 is a schematic representation of current clinical practice for the prognosis and treatment of patients with prostate cancer.

FIG. 3 is a schematic representation of the described method for diagnosing indolent or aggressive prostate cancer.

FIG. 4 is a heatmap representation of ncRNA expression in prostate biopsies clustered by Gleason score for miRNAs, CD Box snoRNAs, and H/ACA box snoRNAs.

FIG. 5A is a progression score plot representation for prostate tissue biopsies analyzed for comparative and cumulative ncRNA expression levels indicating biochemical outcome for the respective patients (i.e., indolent or aggressive) where the patient outcome was known.

FIG. 5B is a progression score plot representation for prostate tissue biopsies analyzed for comparative and cumulative ncRNA expression levels indicating biochemical outcome for the respective patients (i.e., indolent or aggressive) where the patient outcome was unknown.

FIG. 6 is a schematic representation of the benefits of the described method.

FIG. 7 is waterfall plot representations of the progression score calculated using 56 miRNAs and snoRNAs from 38 different patient prostate tissue samples.

FIG. 8 is a schematic representation of the design of stem-loop RT-qPCR of both miRNA and small ncRNA species.

FIG. 9 is a schematic representation of forward and reverse primer and TaqMan® probe design that targeted the limited unique sequence identifier (shaded) of small ncRNA of interest to distinguish it from other highly similar small ncRNAs.

FIG. 10 displays the untransformed data showing the Ct curves for each of the specific miRNA/sncRNA sequences.

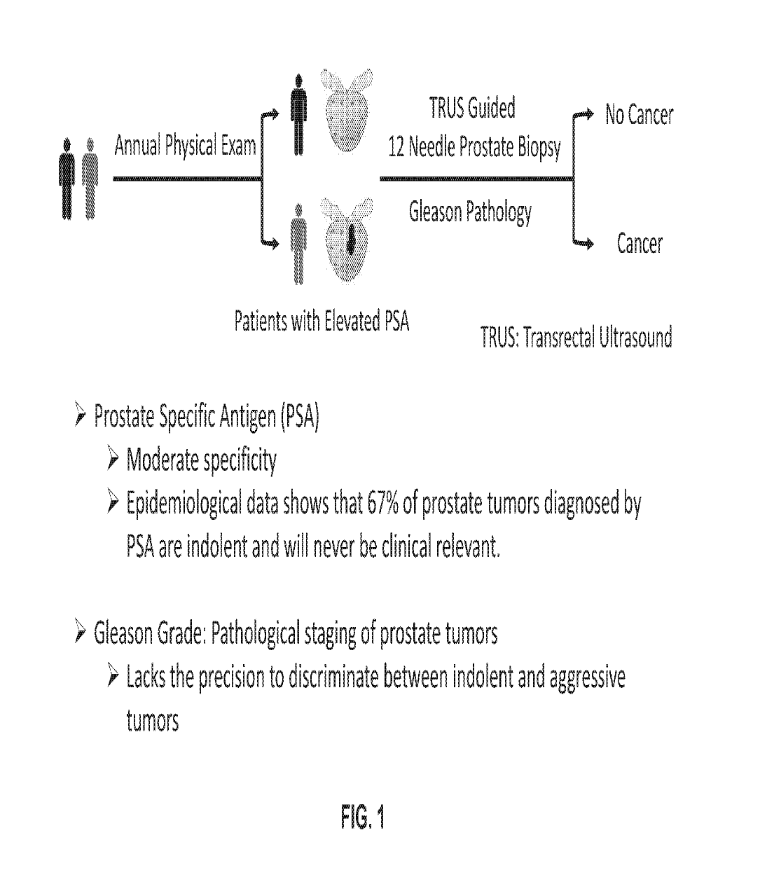

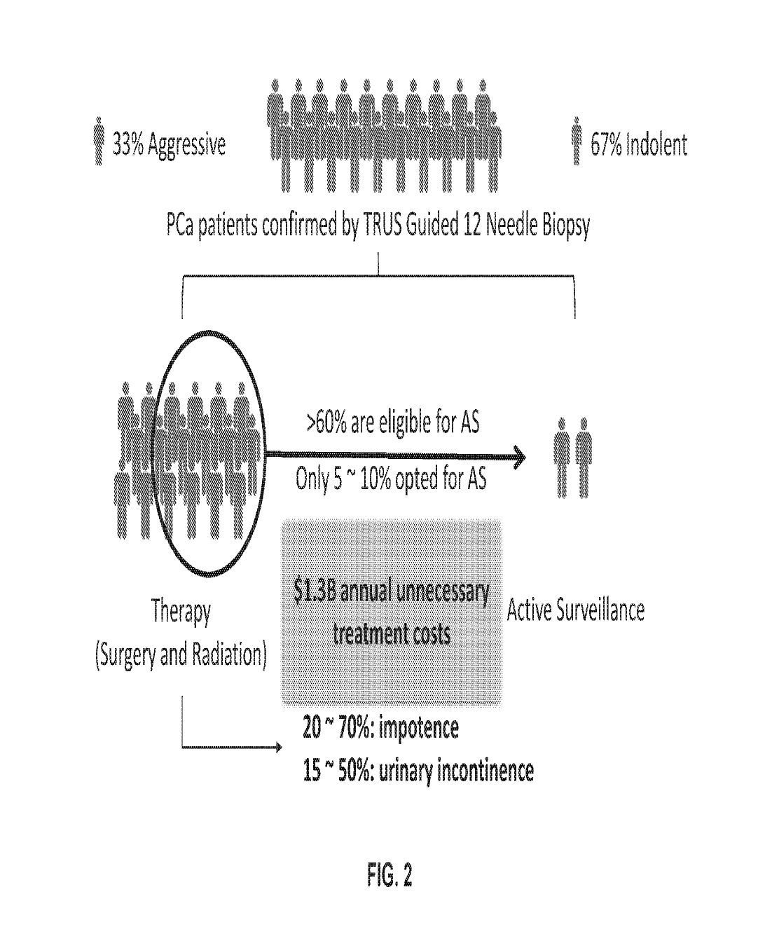

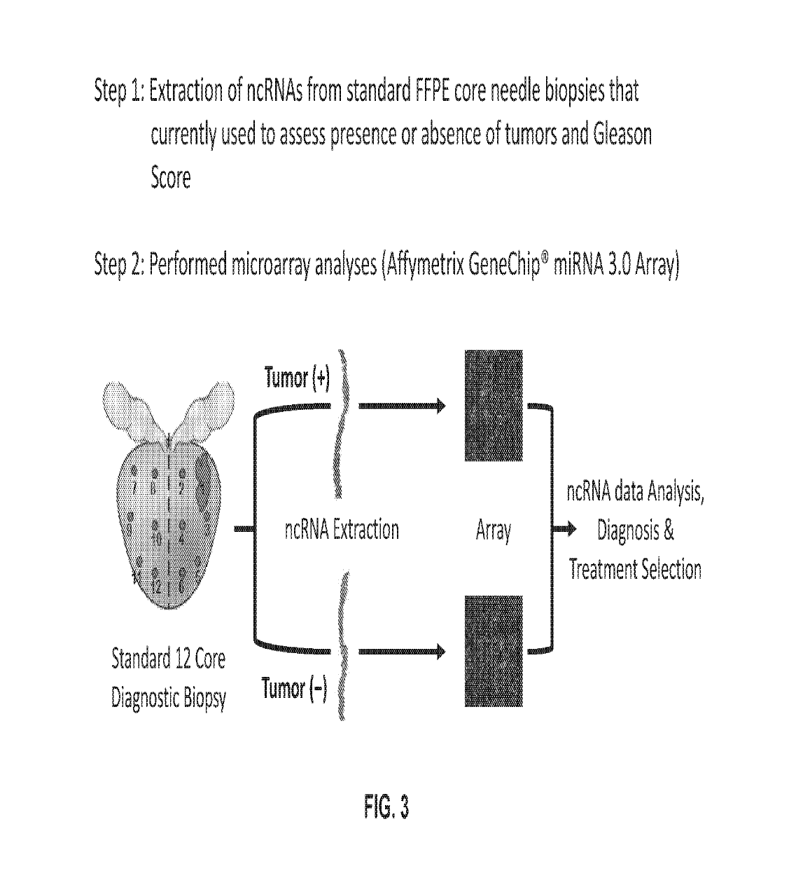

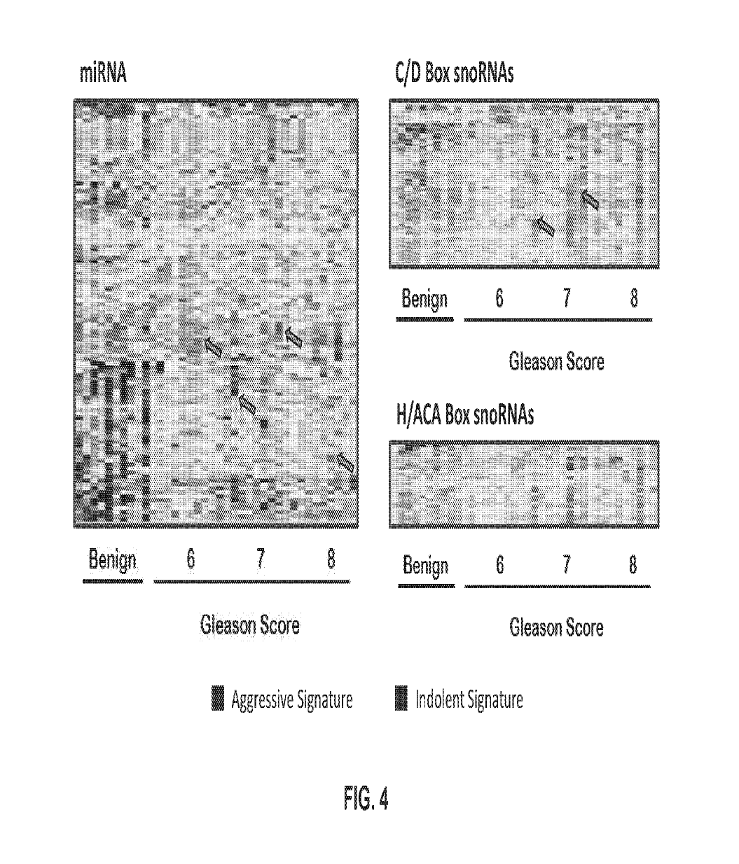

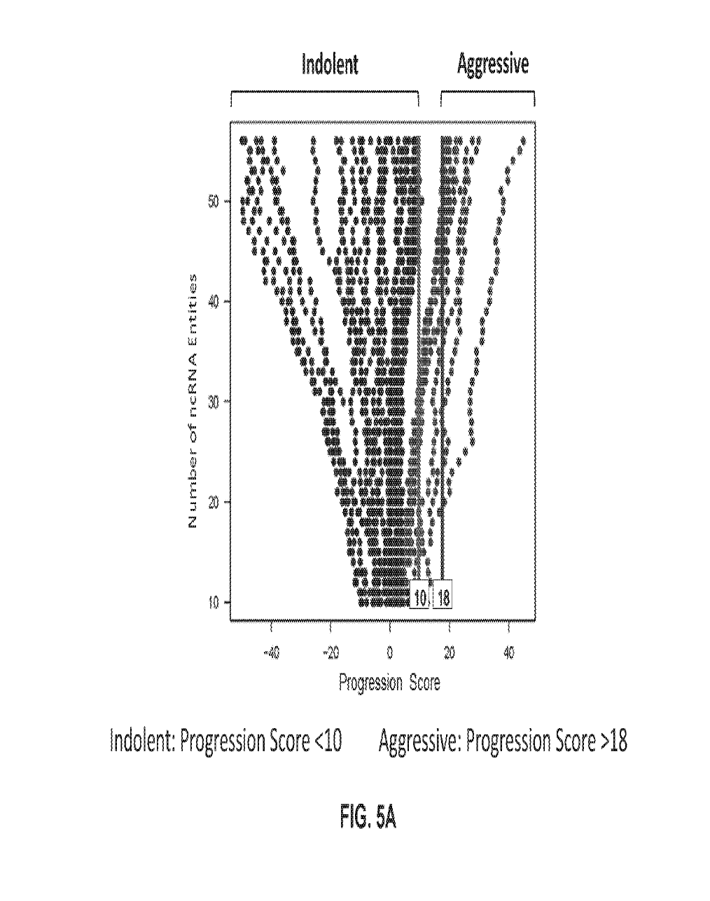

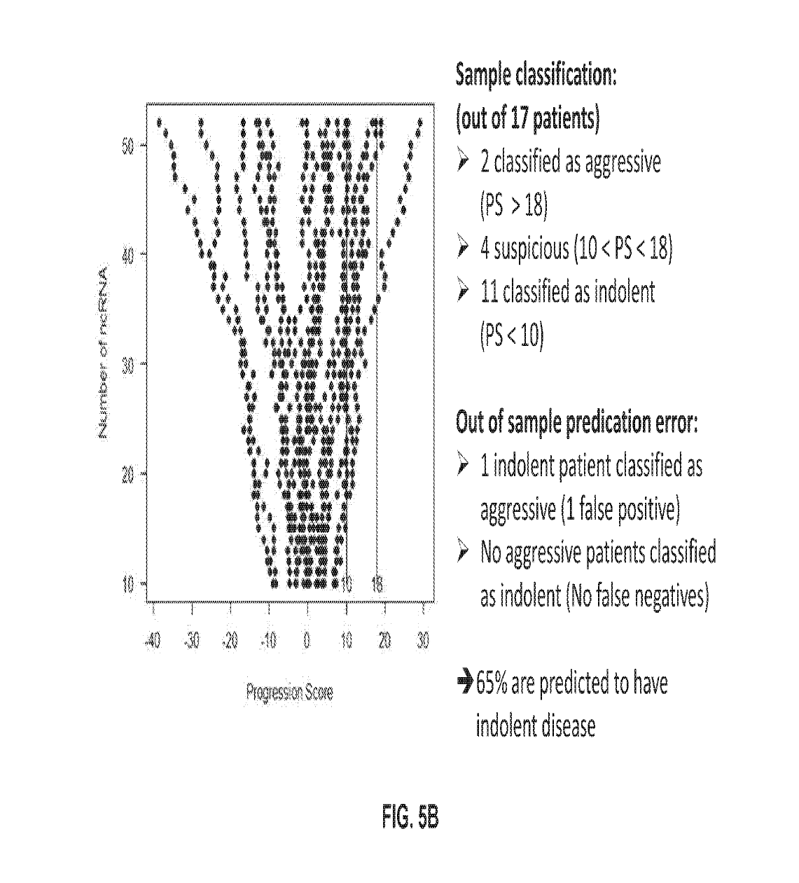

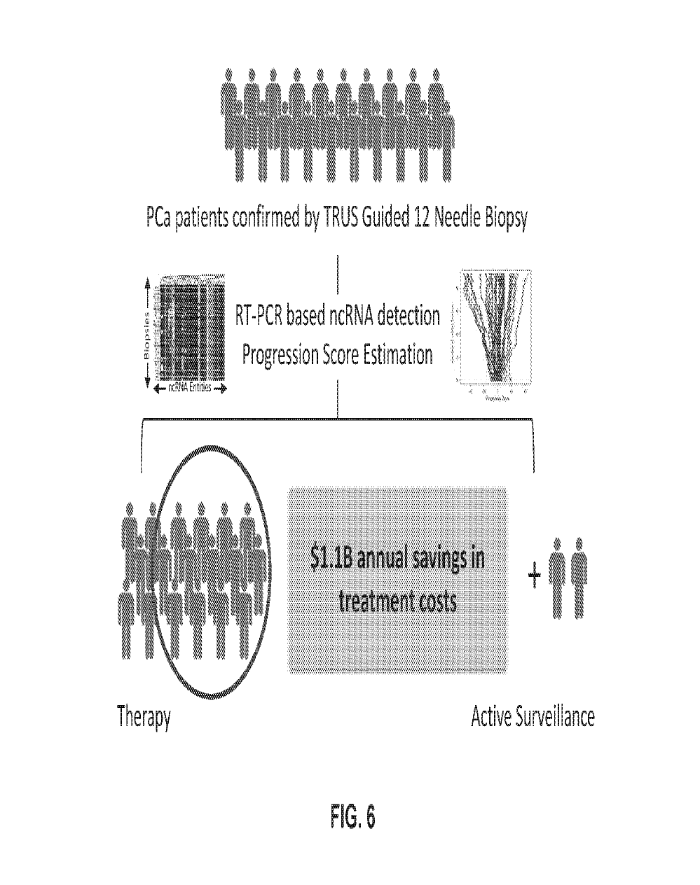

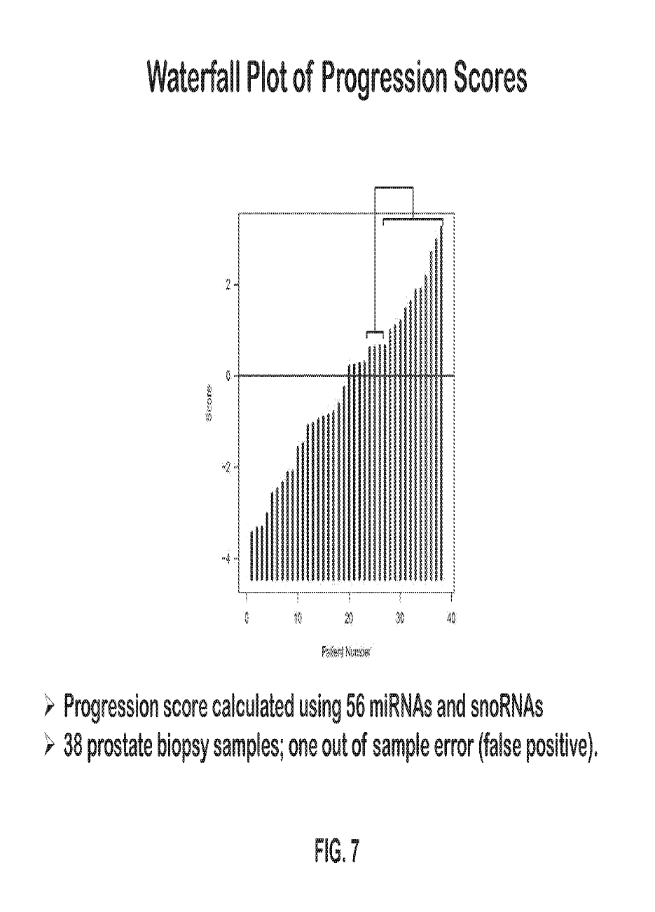

### DETAILED DESCRIPTION OF THE INVENTION

Provided herein is a method for diagnosing indolent or aggressive prostate cancer in a subject. In some embodiments, the method provides a robust Progression Score (PS) to accurately distinguish between indolent and aggressive prostate cancer in biological samples from patients. As used herein, “aggressive” prostate cancer is defined by evidence of biochemical recurrence. Clinically, this includes 1) rising PSA (measured as absolute PSA levels of PSA velocity); 2) evidence of metastatic progression; changes in Gleason Score on re-biopsy (prior to therapy); or evidence of new metastases on X-ray or Catscan. As used herein, “indolent” prostate cancer tumors are defined by the absence of the events of aggressive prostate cancer tumors.

In some embodiments, the method for diagnosing indolent or aggressive prostate cancer in a subject includes obtaining a biological sample from a human patient, detecting the expression level of at least 10 ncRNAs selected from the group consisting of SEQ ID NOs:1-209 by contacting the biological sample with a reagent in an in vitro assay; and identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample.

In some embodiments, the biological sample is selected from the group consisting of prostate tissue, blood, plasma, serum, urine, urine supernatant, urine cell pellet, semen, prostatic secretions, and prostate cells. In particular embodiments, the biological sample is formalin-fixed paraffin-embedded tissue from diagnostic core needle biopsies. In other embodiments, the biological sample is ncRNAs isolated from urinary exosomes.

In certain embodiments, miRNAs (20-24 nucleotides) and ncRNAs (up to 300 nucleotides) are extracted from individual biopsy cores. As used herein, the mixture of small RNAs is referred to collectively as ncRNAs. Due to their size (<300 nucleotides), the ncRNAs are readily extracted from FFPE tissue and are not degraded during fixation or extraction, obviating the problems intrinsic to extraction of mRNA from FFPE tissues. The yield of ncRNAs from the biopsies is sufficient for multiple analyses. Using Affymetrix GeneChip miR 3.0 arrays which contain probes for 5500 small non-coding RNAs (1733 miRNAs, 1658 pre-RNAs, 2216 sno/sca RNAs), we have identified a cohort of ncRNAs that are differentially expressed between benign tissue and Gleason 8 tumors. This analysis has identified miRNAs that target gene ontologies implicated in prostate tumor progression and several ncRNAs (C/D box and H/ACA box snoRNAs) implicated in metastatic progression. In some embodiments, an ncRNA array is produced on a QuantStudio 12K OpenArray™ instrumentation platform, a new highly sensitive, high content, high throughput platform that significantly reduces the cost of the assay.

A select subset of ncRNAs have been identified whose comparative and cumulative expression levels enable distinguishing between indolent and aggressive tumors. The selection of these ncRNAs is independent of PSA, Gleason Score, or biological pathway analysis, and as such is entirely unbiased. An algorithm has been validated using an independent training set that demonstrates that the statistical methodology minimizes both Type 1 (false negative) and Type 2 error (false positive) to ensure that the Progression Score (PS) rigorously distinguishes between indolent and aggressive disease. In its current configuration the method described herein has no false negatives and a very low (<5%) false positive rate.

In some embodiments, the method uses the same OpenArray™ technology to interrogate a panel of ncRNAs (miRNAs, CD/box and HACA/box). In certain embodiments, the method employs an algorithm that relies on the expression level of each of the ncRNAs and the clinical outcome (absence or presence of tumor confined after 12 core needle biopsy for the diagnostic test and biochemical failure and tumor progression for the prognostic test). In the case of prostate cancer, the methodology is independent of serum Prostate Specific Antigen (PSA) levels, Gleason Score (neither of which are meaningful markers of tumor progression). The methodology is also independent of any analyses of biological pathways. Indeed, the methods described herein stratify men into those that have prostate cancer (both early and late) and those that do not, using the analysis of non-coding RNAs isolated from prostate tissue samples. This methodology can replace serum PSA as the major screening assay for prostate cancer.

The methods described herein distinguish indolent from aggressive prostate cancer using the same customized screen, again independent of pathology (Gleason Score), tumor volume or PSA. In some embodiments, RNA extracted from prostate biopsies of patients with known cancer outcomes (i.e., indolent or aggressive) are reverse-transcribed and hybridized against a full-genome array (e.g., Affymetrix GeneChip miR 3.0) containing non-coding RNAs (ncRNAs) and ncRNAs differentially regulated in indolent and aggressive prostate tumors are identified. In contrast to other transcript expression modulation studies or tests, the relative and cumulative expression levels of the identified ncRNAs (SEQ ID NOs:1-209), as compared to the expression profiles found in indolent or aggressive prostate cancers tumors, provide a surprisingly robust and accurate determination of prostate cancer prognosis and, as a result, appropriate treatment options (or lack thereof) can be initiated.

In some embodiments, the relative and cumulative expression profile of at least 10 ncRNAs are combined and compared to the same cumulative expression profile in indolent or aggressive prostate cancer tissue. In certain embodiments, a higher cumulative expression profile as compared to the cumulative expression profile in indolent prostate cancer tissue indicates the patient has aggressive prostate cancer and treatment is required. In other embodiments, a cumulative expression profile equal to or lower than the cumulative expression profile in indolent prostate cancer tissue indicates the patient does not have aggressive prostate cancer and monitoring but not treatment may be required.

In some embodiments, the cumulative expression profile of selected ncRNAs can be an aggregation of various types of modulated expression of the ncRNAs. In some embodiments, the modulated expression can be decreased expression relative to the same ncRNA in other tissue types, such as healthy prostate tissue, indolent prostate cancer tissue, or aggressive prostate cancer tissue. In certain embodiments, the modulated expression can be increased expression relative to the same ncRNA in other tissue types, such as healthy prostate tissue, indolent prostate cancer tissue, or aggressive prostate cancer tissue.

In some embodiments, the cumulative expression profile of selected ncRNAs can be an aggregation of the decreased expression level of certain ncRNAs as well as the increased expression level of other ncRNAs in the same tissue sample. For example, a progression score, or relative cumulative or combined expression level of at least 10 ncRNAs may include one or more ncRNAs with decreases expression levels relative to another tissue type or other ncRNAs in the same tissue sample, while one or more of the remaining at least 10 ncRNAs exhibit increased expression levels relative to another tissue type or other ncRNAs in the same tissue sample. The cumulative expression level of the at least 10 differently modulated ncRNAs, provides a sophisticated, unbiased, indication of whether a prostate cancer tumor is indolent or aggressive. Unlike other methods which merely evaluate the presence or absence, or simple increase or decrease of individual target molecules, as compared to normal tissue, the methods described provide a truly unbiased, independent, and multi-variable analysis of a prostate tissue sample thereby allowing for a surprisingly accurate diagnosis of whether a prostate cancer tumor is indolent or aggressive.

In some embodiments, a prostate tissue sample is analyzed for the relative and cumulative expression profile for at least 10 ncRNAs selected from the group consisting of SEQ ID NOs:1-209, and compared to the relative and cumulative expression profile for the same 10 ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:21, 27, 33, 55, 61, 67, 71, 86, 94, 95, 102, 105, 111, 112, 126, 131, 136, 141, 160, 162, 166, 185, 189, 193, and 202, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:2, 6, 8, 12, 14, 18, 23, 40, 44, 46, 48, 80, 90, 91, 102, 106, 109, 110, 134, 141, 147, 148, 163, 194, and 201, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:9, 20, 24, 25, 27, 30, 31, 39, 41, 42, 47, 50, 54, 55, 60, 67, 83, 94, 97, 103, 108, 122, 168, 195, and 204, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:17, 25, 37, 40, 48, 59, 62, 72, 80, 83, 87, 100, 104, 128, 144, 145, 151, 157, 158, 161, 168, 188, 196, 197, and 209, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:11, 28, 32, 34, 43, 49, 58, 64, 66, 72, 77, 104, 105, 125, 137, 143, 149, 157, 160, 171, 173, 177, 197, 202, and 207, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:13, 15, 19, 33, 37, 38, 57, 63, 71, 76, 81, 84, 85, 89, 95, 112, 129, 131, 135, 146, 150, 155, 160, 200, and 203, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:4, 29, 40, 62, 64, 65, 72, 75, 94, 96, 108, 125, 136, 137, 146, 150, 161, 165, 167, 171, 185, 202, 203, and 209, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:15, 24, 29, 32, 38, 43, 49, 53, 57, 63, 74, 82, 85, 96, 108, 114, 115, 124, 147, 150, 153, 181, 187, 203, and 208, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:7, 12, 20, 22, 23, 39, 47, 51, 60, 64, 69, 89, 90, 91, 121, 134, 138, 142, 145, 146, 148, 150, 155, 161, and 167, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for at least 10 ncRNAs selected from the group consisting of SEQ ID NOs:6, 16, 53, 61, 74, 75, 96, 107, 113, 114, 115, 116, 123, 124, 127, 128, 130, 156, 166, 169, 174, 185, 186, 187, and 190, and, compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 ncRNAs selected from the group consisting of SEQ ID NOs:1-209, and compared to the relative and cumulative expression profile for the same ncRNAs in a prostate tissue sample with indolent cancer or a prostate tissue sample with aggressive cancer.

In some embodiments, a prostate tissue sample is analyzed for the relative and cumulative expression profile for 60-70, 71-80, 81-90, 91-100, 101-110, 111-120, 121-130, 131-140, 141-150, 151-160, 161-170, 171-180, 181-190, 191-200, or 201-209 ncRNAs selected from the group consisting of SEQ ID NOs:1-209, and compared to the relative and cumulative expression profile for the same ncRNAs in a prostate tissue sample with indolent cancer or a prostate tissue sample with aggressive cancer.

Also provided herein is a method of screening a subject for indolent or aggressive prostate cancer. In some embodiments, the method includes hybridizing ncRNAs from a biological sample from the subject with a microarray comprising probes for whole-genome ncRNAs; detecting the relative abundance of hybridization products for at least 10 ncRNAs selected from the group consisting of SEQ ID NOs:1-209; and comparing the cumulative expression levels of the at least 10 ncRNAs from the biological sample with the cumulative expression levels of the at least 10 ncRNAs from an indolent prostate cancer biological sample, wherein an increased level of expression of the at least 10 ncRNAs in the subject is indicative of aggressive prostate cancer in further need of treatment, and wherein an equal or less than level of expression of the at least 10 ncRNAs in the subject is indicative of indolent prostate cancer not in need of further treatment.

Additionally provided herein is a method of treating aggressive prostate cancer in a subject including obtaining a biological sample from a human patient; detecting the expression level of at least 10 ncRNAs selected from the group consisting of SEQ ID NOs:1-209 by contacting the biological sample with a reagent in an in vitro assay; identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample; and treating the aggressive prostate cancer.

In some embodiments, the treating is selected from the group consisting of i) surgery for partial or complete surgical removal of prostate tissue; ii) administering an effective dose of radiation; and iii) administering a therapeutically effective amount of a medication for the treatment of aggressive prostate cancer.

In some embodiments, the detecting of the expression level of at least 10 ncRNAs selected from the group consisting of SEQ ID NOs:1-209 by contacting the biological sample with a reagent in an in vitro assay is provided in a kit. In some embodiments, the kit includes a first reagent solution for isolating ncRNAs from a patient biological sample. In some embodiments, the kit includes a second reagent solution for detecting expression levels of at least 10 ncRNAs from the first reagent solution. In some embodiments, the expression levels are assayed using primer pairs, probes, microarrays, or a combination thereof. In this regard, those of skill in the art will readily recognize that multiple technologies exist for quantitative expression analysis of nucleic acids. In some embodiments, the reagents containing the biological sample are processed using various mechanical and analytical devices such as, but not limited to, centrifuges, thermocyclers, and fluoroimagers.

In certain embodiments, the surgery is chosen from laparoscopic surgery, laparoscopic radical prostatectomy, prostatectomy, and radical retropubic prostatectomy. In other embodiments, the radiation is chosen from external beam radiotherapy, brachytherapy, and particle beam therapy. In some embodiments, the medication for the treatment of aggressive prostate cancer is chosen from a chemotherapeutic and a sex hormone suppressor. In yet other embodiments, the chemotherapeutic is chosen from docetaxel (Taxotere), cabazitaxel (Jetvana), Goserelin (Zoladex), Flutamide (Eulexin), Bicalutamide (Casodex), Abiraterone (Zytiga), and Nilutamide (Nilandron). In some embodiments, the sex hormone suppressor is chosen from Leuprolide (Lupron).

In some embodiments, the likelihood of success or general compatibility of the treatment option is determined by the combined expression level of the selected and analyzed ncRNAs. Personalized medicine is a medical procedure that separates patients into different groups—with medical decisions, practices, interventions and/or products being tailored to the individual patient based on their predicted response or risk of disease. The terms personalized medicine, precision medicine, and stratified medicine also describe this concept of companion therapies. In some embodiments, a combined expression level analysis of at least 10 ncRNAs that is indicative of an aggressive prostate cancer is also indicative of which therapy the subject is most likely to positively respond.

Throughout this specification, unless the context requires otherwise, the word “comprise” or variations such as “comprises” or “comprising” will be understood to imply the inclusion of a stated step or element or integer or group of steps or elements or integers but not the exclusion of any other step or element or integer or group of elements or integers. Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or group of compositions of matter.

Each embodiment described herein is to be applied mutatis mutandis to each and every other embodiment unless specifically stated otherwise. Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features unless specifically stated otherwise.

The present disclosure is not to be limited in scope by the specific embodiments described herein, which are intended for the purpose of exemplification only. Functionally-equivalent products, compositions and methods are clearly within the scope of the disclosure, as described herein.

It is appreciated that certain features of the disclosure, which are, for clarity, described in the context of separate embodiments, can also be provided in combination in a single embodiment. Conversely, various features of the disclosure, which are, for brevity, described in the context of a single embodiment, can also be provided separately or in any suitable sub-combination.

### EXAMPLES

Examples of embodiments of the present disclosure are provided in the following examples. The following examples are presented only by way of illustration and to assist one of ordinary skill in using the disclosure. The examples are not intended in any way to otherwise limit the scope of the disclosure.

### Example 1. Core Needle Biopsy Prognostic Platform for Prostate Cancer

Standard operating protocol for extraction and characterization of miRNA and snoRNA (collectively referred to as ncRNAs) from core needle biopsies. List of informative ncRNA used to develop the prognosis. Waterfall Plot generated from core needle biopsies using the comparative ncRNA expression data set and differentiating between aggressive and indolent tumors.

### Example 2. RNA Extractions from FFRE Core Needle Biopsies

This SOP describes the extraction process and handling of de-identified and barcoded core needle biopsy sections to obtain RNA materials after they are delivered to the laboratory.

The specimen must contain at least two sections of 10 μm thick FFPE tissues from prostate core needle biopsy materials.

### RNA Extraction

### A. Preparation

All laboratory personnel should wear gloves and decontaminate the working environment with appropriate decontaminant when handling or transporting specimens.

RNase, DNase free tubes and filter tips are used when handling RNA materials and during all the extraction procedures. They are properly disposed in biohazard bin and are never reused for any purpose.

Sample tubes, RNA extraction columns and final eluting tubes should either be pre-barcoded or barcoded immediately after RNA materials are obtained.

### B. Procedures

1. Add 1 mL deparaffinization solution or hexadecane and vortex vigorously for 10 s. Briefly centrifuge the tube to bring the sample to the bottom. Remove 840 μL solvent then incubate at 56° C. for 3 min, then allow to cool at room temperature. (If the mixture appears opaque, add additional 1 mL of deparaffinization solution or hexadecane to dissolve excess paraffin. Remove excess solvent to reach final volume of 160 μL).

2. Add 150 μL Buffer PKD to sample tube, vortex briefly and centrifuge for 1 min at 11,000 g.

3. Add 10 μL proteinase K to the lower, clear phase. Mix by gentle pipetting.

4. Incubating at 56° C. for 15 min, then 80° C. for 15 min.

5. Transfer the lower, clear phase into a new 2 mL microcentrifuge tube and incubate on ice for 3 min. Centrifuge for 15 min at 20,000 g. For robotic purification with QIACube, transfer the supernatant into 2 mL sample tube RB and place the tubes in QIACube.

6. Add 16 μL DNase Booster Buffer and 10 μL DNase I stock solution. Mix by inverting the tubes. Centrifuge briefly.

7. Incubate at room temperature for 15 min, and then add 320 μL Buffer RBC. Pipette to mix.

8. Add 1120 μL 100% EtOH to the sample, mix well by pipetting.

9. Transfer 700 μL of the sample to RNeasy MiniElute spin column and close the lid. Either, i) switch on the vacuum that the spin columns attached to; apply vacuum is complete; switch off the vacuum and ventilate the vacuum manifold; ii) centrifuge for 30 s ≥8000 g; discard flow-through. (Repeat till all sample has passed through the spin column).

10. Add 500 μL Buffer RPE to the spin column and close the lid. Either, i) switch on the vacuum; apply vacuum until transfer is complete; switch off the vacuum and ventilate the vacuum manifold; ii) centrifuge for 30 s at ≥8000 g; discard flow-through.

11. Add 500 μL Buffer RPE to the spin column and close the lid. Switch on the vacuum. Apply vacuum until transfer is complete. Switch off the vacuum and ventilate the vacuum manifold. Centrifuge for 2 min at ≥8000 g.

12. Place the spin column in a new 2 mL collection tube. Centrifuge at full speed for 5 in with the lid open.

13. Place the spin column in a new pre-barcoded 1.5 mL collection tube. Add 20 μL RNase-free water directly to the spin column membrane. Close the lid and centrifuge for 1 min at full speed to elute RNA.

### Post-Extraction RNA Handling

1. Determine the concentration of total RNA obtained from each prostate core needle biopsy materials using NanoDrop spectrophotometers with 1.5 μL of final elute.

2. RNA materials should be transferred and stored in pre-barcoded 96-well RNase, DNase free plate with matching accession number and QC summary corresponding to each well, stored on private server for usage in electronic notebook.

3. All samples should be stored in designated −80° C. freezer till ready for analysis.

### Example 3. QIAcube Automated System for RNA Extraction from FFPE Tissues

This SOP describes the setup and procedure to run QIAcube automated system for total RNA purification from FFPE tissue sections.

The material must contain at least two sections of 10 μm thick FFPE tissues from prostate core needle biopsy materials that have been deparaffinized, proteinase K digested and free of insoluble tissues and materials.

### RNA Extraction

### A. Preparation

All laboratory personnel should wear gloves and decontaminate the working environment with appropriate decontaminant when handling or transporting specimens.

Pre-barcoded 1.5 mL RNase, DNase free collection tubes and 1000 μL filter tips (Qiagen Cat#990352) must be used when working with Qiacube. After use, they are properly disposed in biohazard bin and are never reused for any purpose.

Fill up all the reagent bottles need for RNA extract, including Buffer RBC, 100% ethanol, Buffer RPE (with 100% ethanol added) and RNase, DNase-free water.

Check the nozzle ring on the instrument before start of the run.

### B. Procedures

1. Prepare DNase I incubation mix for 12 samples (145 μL DNase I mix+232 μL DNase Booster Buffer) in 2 mL RB sample tube. Place the tube in the microcentrifuge tube slot A.

2. Place Buffer RBC in position 2, 100% ethanol in position 3, Buffer RPE in position 5 and RNase, DNase-free water in position 6 of the reagent bottle rack. Open the cap and place onto QIAcube.

3. Place two full racks of disposable 1000 μL filter-tips in QIAcube.

4. Set up the Rotor Adapter from 1 to 12 on the rotor adapter holder. i) place RNeasy® MinElute® spin column in position 1 and bend the lid and fit in LI position; ii) place pre-barcoded 1.5 mL collection tube in position 3 and bend the lid and fit in L3 position.

5. Place the rotor adapter into the centrifuge with matched number as the one on the rotor adapter holder.

6. Transfer samples to the 2 mL RB sample tubes and place onto the QIAcube with matching numerical number as the ones in the centrifuge and rotor adapter holder. Bend the lid and place them in lid position, provided in the sample tray.

7. Close the cover and start the running protocol for miRNeasy FFPE purification with 1-2 sections of 10 μm FFPE tissues.

### Post-Extraction RNA Handling and QIAcube Care

1. Remove all sample tubes RB, including the one with DNase I incubation mix and filter tips from the trash bin into the biohazard bin.

2. Remove the reagent bottle rack, cap all the bottles for storage.

3. Remove Rotor Adapter from the centrifuge. Close the caps of the collection tubes with the final RNA elutes and store at −80° C. till ready for spec. Dispose the spin columns into the biohazard bin and discard the flow thru into the waste bucket labeled “guanidine thiocyanate”. Dispose the Rotor Adapter into the biohazard bin.

4. Determine the concentration of total RNA obtained from each prostate core needle biopsy materials using NanoDrop spectrophotometers with 1.5 μL of final elute.

5. RNA materials should be transferred and stored in pre-barcoded 96-well RNase, DNase free plate with matching accession number and QC summary corresponding to each well, stored on private server for usage in electronic notebook.

6. All samples should be stored in designated −80° C. freezer till ready for analysis.

### Example 4. Assay Design

Recent advances in molecular biology have uncovered new roles for small non-coding RNAs in many human diseases, and it has become apparent that combined effects of multiple types of RNA contribute to the etiology of human diseases. This warrants the development of new assays that are capable of interrogating multiple RNA species in clinical samples.

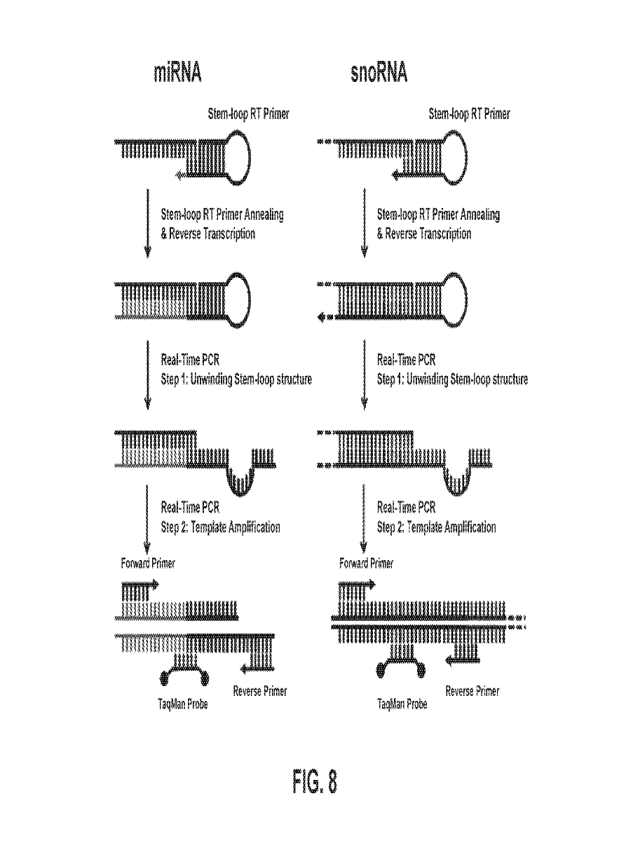

It is possible to combine the detection and analysis of two different types of RNA species into a single platform for simultaneous detection and analysis (FIG. 8). This in turn amplifies data output by RT-qPCR assays without compromising data integrity of either type of RNA. This technology can be further adapted into high-throughput platform to match the demand in clinical applications. However, few of the available assay design platforms, such as the ones used by Thermo Fisher only uses the last 60 nucleotides of any small RNA sequence provided for customized assay design, which sometimes lack specificity. It is therefore important to validate the assay specificity by identifying unique sequence identifiers for each small ncRNAs of interest when designing the stem-loop RT-qPCR.

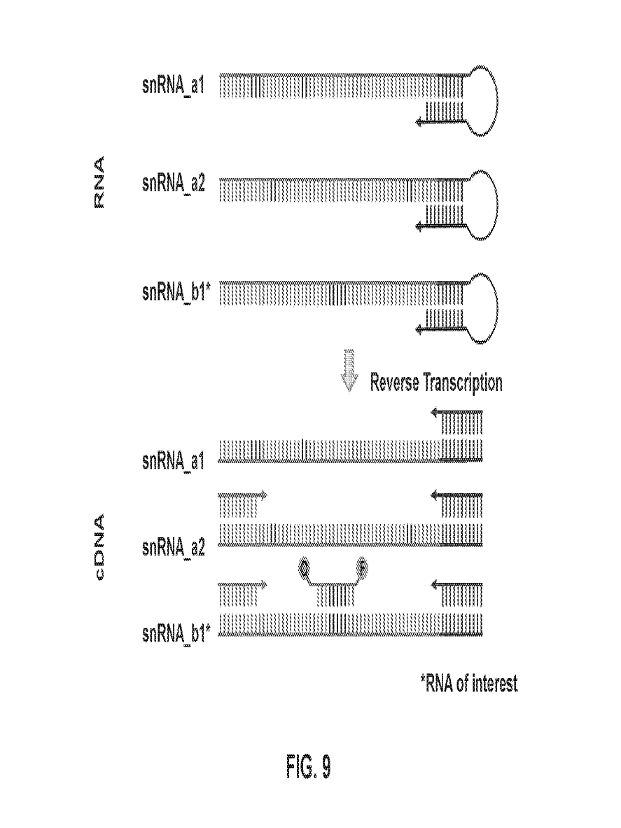

Use of the last 60 nucleotides at the 3′ end of small ncRNA sequence of interest for stem-loop reverse transcription primer design, either through Thermo Fisher Scientific Custom TaqMan® Small RNA Assay Design Tool or by miRNA Primer Design Tool (Astrid Research, Inc.). The entire sequence is used after cDNA synthesis for RT-qPCR primer and TaqMan® probe design, using Primer3 software (v.0.4.0.) with default parameter (Primer size: 18-27nts, Optimal 20 nts; Primer Tm: 57-63° C.; Primer GC %: 20%-80%; PCR product size range: 60-130 nts). The specificity of the primer pairs to one target was verified using Primer-Blast. If the small ncRNA sequence of interest shares high sequence homology with other ncRNAs, i.e., the C/D Box and H/ACA Box small nucleolar RNAs, regions of unique sequence must be identified to select primers and/or a TaqMan® probe to maximize specificity (FIG. 9). All custom designed small ncRNA assays are validated by sequencing.

### Example 5. Reverse Transcription and Affymetrix/OpenArray MicroArray Hybridization and Expression Profiling

This SOP describes the preparation and running of RNA materials obtained from de-identified and barcoded core needle biopsy sections for RT-PCR based analysis, run on the OpenArray™ platform by QuantStudio™ 12K Flex Real-Time PCR system. While the OpenArray™ technology allows for customized arrays with specific hybridization targets, the various Affymetrix arrays are sold commercially with preloaded hybridization targets.

The specimens are total RNAs obtained from core needle biopsy sections that are previously de-identified and processed as above.

### RT-PCR on OpenArray Platform

### A. Preparation

All laboratory personnel should wear gloves and decontaminate the working environment with appropriate decontaminant when handling or transporting specimens.

RNase, DNase free tubes, plates and filter tips are used when handling RNA materials and during all procedures. Used tubes and tips are properly disposed in biohazard bin and are never reused for any purpose.

Tubes and 96 well plates should be pre-barcoded or barcoded immediately after sample materials have been transferred into and correctly correspond to the previously assigned barcode.

All of the preparation process needs to be performed and stored on ice with cooling blocks till ready to be run on the PCR machine

### B. Procedures

I. cDNA Synthesis

1. Dilute RNA extracted from de-identified biopsy materials into 50 ng/3 μL with DNase and RNase free H₂O in pre-barcoded 96 well plate. Sample information and accession number will be coordinately logged and stored in excel format for each individual plate for future tracking. Samples within the same 96 well plate is considered as a working batch.

2. Prepare RT master mix for cDNA synthesis as followed (Enough for 96 reactions needed):

| 1 rxn | 96 rxn | (w/10% extra) |
|---|---|---|
| 0.75 | 79.20 | μL Custom RT Primers (10X) |
| 0.15 | 15.84 | μL dNTPs with dTTP (100 mM) |
| 1.50 | 158.4 | μL MultiScribe ™ Reverse Transcriptase (50U/μL) |
| 0.75 | 79.20 | μL 10X RT Buffer |
| 0.90 | 95.04 | μL MgCl2 (25 mM) |
| 0.09 | 9.50 | μL RNase Inhibitor (20 U/μL) |
| 0.36 | 38.02 | μL Nuclease-free water |

3. Load 4.5 μL of RT master mix into pre-barcoded PCR grade (DNase, RNase, Pyrogen, DNA and RNA free) 96 well plate. Add 3 μL of diluted total RNA into each well and mix gently by pipetting. Seal the plate using PCR grade and sterile adhesive foil, and spin briefly.

4. Incubate the reaction plate on ice for 5 minutes prior to loading into the PCR machine to perform reverse transcription run as shown in the table below.

| Temperature (° C.) | Time | |
|---|---|---|
| 16.0 | 2:00 | 40 cycles |
| 42.0 | 1:00 | |
| 50.0 | 0.01 | |
| 85.0 | 5:00 | Hold |
| 4.0 | ∞ | Hold |

II. Pre-Amplification

1. Prepare the PreAmp master mix for the Pre-Amplification cycles as followed (Enough for 96 samples):

| 1 rxn | 96 rxn | (w/10% extra) |
|---|---|---|
| 12.5 | 1320 | μL Custom PreAmp Primers (10X) |
| 2.5 | 264 | μL TaqMan PreAmp Master Mix (2X) |
| 7.5 | 792 | μL Nuclease-free H2O |

2. Load 22.5 μL PreAmp mast mix into pre-barcoded PCR grade (DNase, RNase, Pyrogen, DNA and RNA free) 96 well plate. Add 2.5 μL of cDNA into each well. Seal the plate with PCR grade and sterile adhesive foil, mix by inverting the plate 6 times. Spin briefly.

3. Incubate the reaction plate on ice for 5 minutes prior to loading into the PCR machine to perform pre-amplification cycles as shown in the below.

| Temperature (° C.) | Time | |
|---|---|---|
| 95.0 | 10:00 | Hold |
| 55.0 | 2:00 | Hold |
| 72.0 | 2:00 | Hold |
| 95.0 | 0.15 | 14 cycles |
| 60.0 | 4:00 | |
| 99.9 | 10:00 | Hold |
| 4.0 | ∞ | Hold |

III. OpenArray

1. Add 152 μL of 0.1×TE buffer to PCR grade 96-well plate. Transfer 8 μL of pre-amplification product to each well to make 1:20 dilution.

2. Seal the 96-well plate with PCR grade and sterile foil and mix by inverting the plate six times. Centrifuge the plate briefly.

3. Warm the OpenArray™ plate to room temperature for 30 minutes and follow the instruction to load OpenArray™ plate one by one. Assemble four loaded and sealed OpenArray™ plates on the carrier tray and insert into QuantStudio 12K Flex system to perform Real-Time PCR stage.

4. Collect raw data and analyze with ExpressionSuite software to obtain relative expression level. Upload the result to the secure miR Diagnostic cloud for future analysis.

IV. Post RT-PCR Run Sample Handling

Store all of the initial RNA materials with proper QR code and all working batch 96-well plates from cDNA synthesis, pre-amplification and diluted pre-amplification cycle in designated −80° C.

### Example 6. Interrogation of miRNA Using a Custom Designed 56 miRNA Customized Open Array in the Quant Studio 12K Flex

In Example 6, probes for 56 Sentinel miRNAs were pre-loaded onto customized Open Array plates, providing 48 identical samples wells designed to interrogate 56 specific miRNAs.

The snRNA was prepared from patient samples described previously. cDNA was synthesized as described above, and 500 ng from each cDNA was loaded into individual wells.

In these described experiments. the probes used were labeled with FAM at the 5′-end and the respective reporter/quencher was TAMRA at the 3′-end.

The digital PCR was set for 40 cycles

During each cycle, successful amplification of the cDNA caused probe displacement and cleavage from each copy of target in the specific well, resulting in an increase in fluorescence. The camera/detector reports total amount of fluorescence at the end of each cycle in each well.

The raw data are probe and cycle number dependent. Raw fluorescent data readings are retrieved from each well, for each cycle, and for each biological sample. (see Table 1).

Change in fluorescence relative to previous cycle is given (ΔRn) and transposed to provide relative changes in fluorescence for each cycle. The data are subsequently sorted by clinical descriptors, i.e., patient number and core number, in this example.

### Example 7. Interrogation of Mixed miRNA and snoRNAs Using the 384 Well Block in the Quant Studio 12K Flex

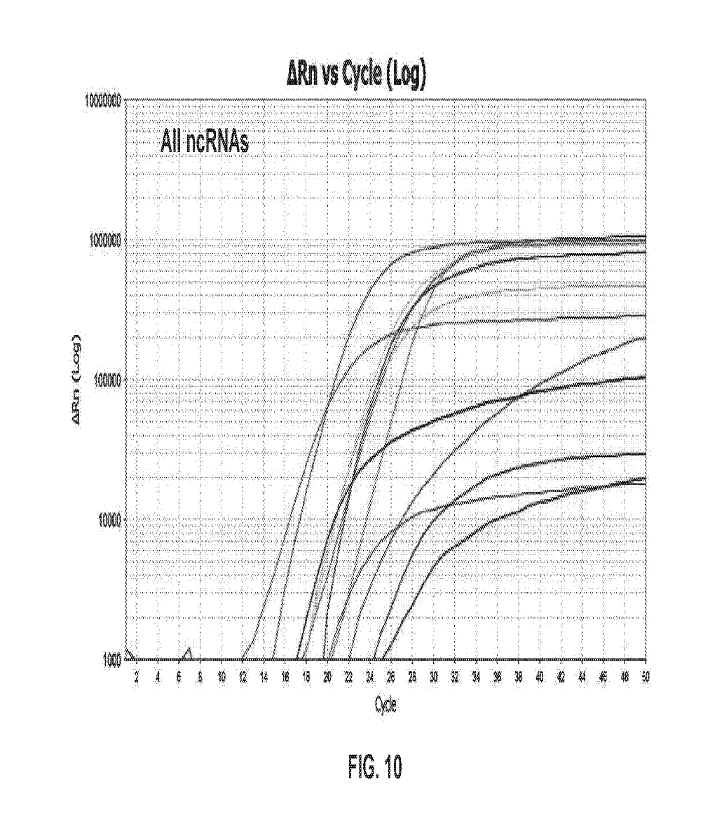

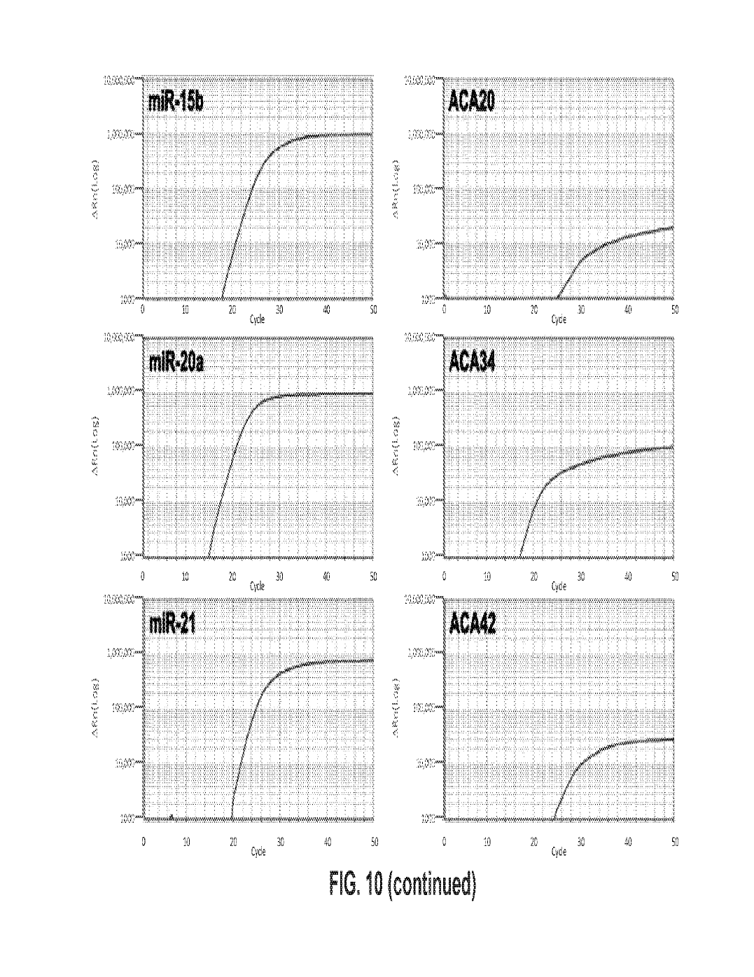

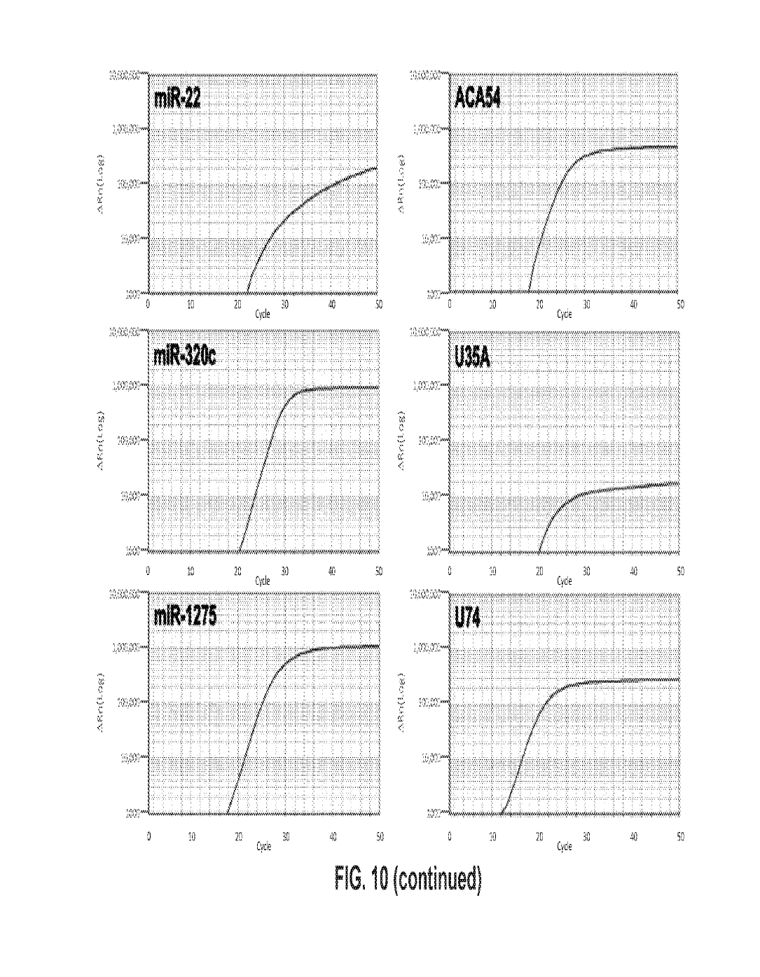

The ability of the assay to efficiently interrogate a mix miRNAs and small ncRNAs has been validated using a testing set 6 microRNAs (miR15b, miR20a, miR21, miR22, miR320c and miR1275) and 6 sncRNAs (4 H/ACA box snoRNAs (ACA20, ACA34, ACA42, ACA54 and 2 C/D box snoRNAs U35A and U74). The cDNAs for these RNA species were reverse transcribed and amplified as described in the Examples herein, in a single RT mix using gene specific primers as described herein. The same reverse transcription cDNA products were used and successfully detected each individual miRNA and ncRNA with their own specific RT-qPCR assay mixture. The signal acquisition was as described in Example 6. The plot in FIG. 10 displays the untransformed data showing the Ct curves for each of the specific miRNA/sncRNA sequences. The transformation of the data to determine the time to event eliminates the issues related to the determination of the Ct for probes with slightly different hybridization kinetics.

For the experiments, the TaqMan probe contains the FAM fluorophore and TAMRA as the quencher. TAMRA may be substituted by MGB (minor groove binder) to enhance the specificity and sensitivity of the assay.

The choice of fluorophore and quencher is slightly dependent on the platform used. The choice of fluorophore can be extended depending on excitation lasers available and the sensitivity of the detection system/camera to the emission wavelength.

### Example 8. ncRNA Sequences

TABLE 1
ncRNA Sequences (SEQ ID NO: 1-SEQ ID NO: 209)

| SEQ ID NO: | Sequence Identifer | Sequence |
|---|---|---|
| SEQ ID NO: 1 | MIR1263 | CUACCCCAAAAUAUGGUACCCUGGCAUAC |
| | | UGAGUAUUUUAAUACUGGCAUACUCAGUA |
| | | UGCCAUGUUGCCAUAUUUUGGGGUAGCA |
| SEQ ID NO: 2 | hsa-miR-320c-1 | UUUGCAUUAAAAAUGAGGCCUUCUCUUCC |
| | | CAGUUCUUCCCAGAGUCAGGAAAAGCUGG |
| | | GUUGAGAGGGUAGAAAAAAAAUGAUGUA |
| | | GG |
| SEQ ID NO: 3 | hsa-miR-4449 | AGCAGCCCUCGGCGGCCCGGGGGGCGGGC |
| | | GGCGGUGCCCGUCCCGGGGCUGCGCGAGG |
| | | CACAGGCG |
| SEQ ID NO: 4 | hsa-miR-4679-1 | GUCUUUUUUCUGUGAUAGAGAUUCUUUGC |
| | | UUUGUUAGAAACAAAAAGCAAAGAAUCUC |
| | | UAUCACAGAAAAAAGAU |
| SEQ ID NO: 5 | hsa-miR-520h | UCCCAUGCUGUGACCCUCUAGAGGAAGCA |
| | | CUUUCUGUUUGUUGUCUGAGAAAAAACAA |
| | | AGUGCUUCCCUUUAGAGUUACUGUUUGGG |
| | | A |
| SEQ ID NO: 6 | hsa-miR-548ai | GUAUUAGGUUGGUGCAAAGGUAAUUGCA |
| | | GUUUUUCCCAUUUAAAAUAUGGAAAAAAA |
| | | AAUCACAAUUACUUUUGCAUCAACCUAAU |
| | | AA |
| SEQ ID NO: 7 | hsa-let-7c-5p | UGAGGUAGUAGGUUGUAUGGUU |
| SEQ ID NO: 8 | hsa-let-7f-5p | UGAGGUAGUAGAUUGUAUAGUU |
| SEQ ID NO: 9 | hsa-let-7g-5p | UGAGGUAGUAGUUUGUACAGUU |
| SEQ ID NO: 10 | hsa-let-7i-5p | UGAGGUAGUAGUUUGUGCUGUU |
| SEQ ID NO: 11 | hsa-miR-106b-3p | CCGCACUGUGGGUACUUGCUGC |
| SEQ ID NO: 12 | hsa-miR-10b | UACCCUGUAGAACCGAAUUUGUG |
| SEQ ID NO: 13 | hsa-miR-1180-3p | UUUCCGGCUCGCGUGGGUGUGU |
| SEQ ID NO: 14 | hsa-miR-125a-3p | ACAGGUGAGGUUCUUGGGAGCC |
| SEQ ID NO: 15 | hsa-mir-125b-1-3p | ACGGGUUAGGCUCUUGGGAGCU |
| SEQ ID NO: 16 | hsa-miR-125b-2-3p | UCACAAGUCAGGCUCUUGGGAC |
| SEQ ID NO: 17 | hsa-miR-1260a | AUCCCACCUCUGCCACCA |
| SEQ ID NO: 18 | hsa-mir-1263 | AUGGUACCCUGGCAUACUGAGU |
| SEQ ID NO: 19 | hsa-miR-127-3p | UCGGAUCCGUCUGAGCUUGGCU |
| SEQ ID NO: 20 | hsa-miR-1272 | GAUGAUGAUGGCAGCAAAUUCUGAAA |
| SEQ ID NO: 21 | hsa-miR-1281 | UCGCCUCCUCCUCUCCC |
| SEQ ID NO: 22 | hsa-miR-1296-5p | UUAGGGCCCUGGCUCCAUCUCC |
| SEQ ID NO: 23 | hsa-miR-1301-3p | UUGCAGCUGCCUGGGAGUGACUUC |
| SEQ ID NO: 24 | hsa-miR-130b | CAGUGCAAUGAUGAAAGGGCAU |
| SEQ ID NO: 25 | hsa-miR-132 | UAACAGUCUACAGCCAUGGUCG |
| SEQ ID NO: 26 | hsa-miR-134-5p | UGUGACUGGUUGACCAGAGGGG |
| SEQ ID NO: 27 | hsa-miR-141-3p | UAACACUGUCUGGUAAAGAUGG |
| SEQ ID NO: 28 | hsa-miR-145-5p | GUCCAGUUUUCCCAGGAAUCCCU |
| SEQ ID NO: 29 | hsa-miR-146a-5p | UGAGAACUGAAUUCCAUGGGUU |
| SEQ ID NO: 30 | hsa-mir-146-5p | UGAGAACUGAAUUCCAUAGGCU |
| SEQ ID NO: 31 | hsa-miR-148a-3p | UCAGUGCACUACAGAACUUUGU |
| SEQ ID NO: 32 | hsa-miR-150-5p | UCUCCCAACCCUUGUACCAGUG |
| SEQ ID NO: 33 | hsa-miR-151a-3p | CUAGACUGAAGCUCCUUGAGG |
| SEQ ID NO: 34 | hsa-miR-155-5p | UUAAUGCUAAUCGUGAUAGGGGU |
| SEQ ID NO: 35 | hsa-miR-15a-5p | UAGCAGCACAUAAUGGUUUGUG |
| SEQ ID NO: 36 | hsa-miR-17-3p | ACUGCAGUGAAGGCACUUGUAG |
| SEQ ID NO: 37 | hsa-miR-181a-2-3p | ACCACUGACCGUUGACUGUACC |
| SEQ ID NO: 38 | hsa-miR-181b-5p | AACAUUCAUUGCUGUCGGUGGGU |
| SEQ ID NO: 39 | hsa-miR-182-5p | UUUGGCAAUGGUAGAACUCACACU |
| SEQ ID NO: 40 | hsa-miR-18a-5p | UAAGGUGCAUCUAGUGCAGAUAG |
| SEQ ID NO: 41 | hsa-miR-193b-3p | AACUGGCCCUCAAAGUCCCGCU |
| SEQ ID NO: 42 | hsa-miR-195-3p | CCAAUAUUGGCUGUGCUGCUCC |
| SEQ ID NO: 43 | hsa-miR-197-3p | UUCACCACCUUCUCCACCCAGC |
| SEQ ID NO: 44 | hsa-miR-199a-5p | CCCAGUGUUCAGACUACCUGUUC |
| SEQ ID NO: 45 | hsa-miR-202-3p | AGAGGUAUAGGGCAUGGGAA |
| SEQ ID NO: 46 | hsa-miR-21-5p | UAGCUUAUCAGACUGAUGUUGA |
| SEQ ID NO: 47 | hsa-miR-25-3p | CAUUGCACUUGUCUCGGUCUGA |
| SEQ ID NO: 48 | hsa-miR-2861 | GGGGCCUGGCGGUGGGCGG |
| SEQ ID NO: 49 | hsa-mir-29b-2-5p | CUGGUUUCACAUGGUGGCUUAG |
| SEQ ID NO: 50 | hsa-miR-29c-3p | UAGCACCAUUUGAAAUCGGUUA |
| SEQ ID NO: 51 | hsa-miR-30a-3p | CUUUCAGUCGGAUGUUUGCAGC |
| SEQ ID NO: 52 | hsa-miR-30b-5p | UGUAAACAUCCUACACUCAGCU |
| SEQ ID NO: 53 | hsa-miR-30d-5p | UGUAAACAUCCCCGACUGGAAG |
| SEQ ID NO: 54 | hsa-miR-320e | AAAGCUGGGUUGAGAAGG |
| SEQ ID NO: 55 | hsa-miR-324-3p | ACUGCCCCAGGUGCUGCUGG |
| SEQ ID NO: 56 | hsa-miR-331-3p | GCCCCUGGGCCUAUCCUAGAA |
| SEQ ID NO: 57 | hsa-miR-337-5p | GAACGGCUUCAUACAGGAGUU |
| SEQ ID NO: 58 | hsa-miR-338-3p | UCCAGCAUCAGUGAUUUUGUUG |
| SEQ ID NO: 59 | hsa-miR-339-3p | UGAGCGCCUCGACGACAGAGCCG |
| SEQ ID NO: 60 | hsa-miR-339-5p | UCCCUGUCCUCCAGGAGCUCACG |
| SEQ ID NO: 61 | hsa-mir-342-5p | AGGGGUGCUAUCUGUGAUUGA |
| SEQ ID NO: 62 | hsa-miR-345-5p | GCUGACUCCUAGUCCAGGGCUC |
| SEQ ID NO: 63 | hsa-miR-3609 | CAAAGUGAUGAGUAAUACUGGCUG |
| SEQ ID NO: 64 | hsa-miR-3615 | UCUCUCGGCUCCUCGCGGCUC |
| SEQ ID NO: 65 | hsa-miR-362-5p | AAUCCUUGGAACCUAGGUGUGAGU |
| SEQ ID NO: 66 | hsa-miR-363-5p | CGGGUGGAUCACGAUGCAAUUU |
| SEQ ID NO: 67 | hsa-miR-363-3p | AAUUGCACGGUAUCCAUCUGUA |
| SEQ ID NO: 68 | hsa-miR-3651 | CAUAGCCCGGUCGCUGGUACAUGA |
| SEQ ID NO: 69 | hsa-miR-3679-5p | UGAGGAUAUGGCAGGGAAGGGGA |
| SEQ ID NO: 70 | hsa-miR-3687 | CCCGGACAGGCGUUCGUGCGACGU |
| SEQ ID NO: 71 | hsa-miR-375 | UUUGUUCGUUCGGCUCGCGUGA |
| SEQ ID NO: 72 | hsa-miR-378a-5p | CUCCUGACUCCAGGUCCUGUGU |
| SEQ ID NO: 73 | hsa-miR-378i | ACUGGACUAGGAGUCAGAAGG |
| SEQ ID NO: 74 | hsa-miR-379-5p | UGGUAGACUAUGGAACGUAGG |
| SEQ ID NO: 75 | hsa-miR-382-5p | GAAGUUGUUCGUGGUGGAUUCG |
| SEQ ID NO: 76 | hsa-miR-3940-5p | GUGGGUUGGGGCGGGCUCUG |
| SEQ ID NO: 77 | hsa-miR-409-3p | GAAUGUUGCUCGGUGAACCCCU |
| SEQ ID NO: 78 | hsa-miR-423-3p | AGCUCGGUCUGAGGCCCCUCAGU |
| SEQ ID NO: 79 | hsa-miR-424-3p | CAAAACGUGAGGCGCUGCUAU |
| SEQ ID NO: 80 | hsa-miR-425-5p | AAUGACACGAUCACUCCCGUUGA |
| SEQ ID NO: 81 | hsa-miR-4270 | UCAGGGAGUCAGGGGAGGGC |
| SEQ ID NO: 82 | hsa-miR-4284 | GGGCUCACAUCACCCCAU |
| SEQ ID NO: 83 | hsa-miR-4286 | ACCCCACUCCUGGUACC |
| SEQ ID NO: 84 | hsa-miR-432-5p | UCUUGGAGUAGGUCAUUGGGUGG |
| SEQ ID NO: 85 | hsa-miR-4417 | GGUGGGCUUCCCGGAGGG |
| SEQ ID NO: 86 | hsa-miR-4449 | CGUCCCGGGGCUGCGCGAGGCA |
| SEQ ID NO: 87 | hsa-miR-4485-3p | UAACGGCCGCGGUACCCUAA |
| SEQ ID NO: 88 | hsa-mir-4516 | GGGAGAAGGGUCGGGGC |
| SEQ ID NO: 89 | hsa-miR-4668-5p | AGGGAAAAAAAAAAGGAUUUGUC |
| SEQ ID NO: 90 | hsa-miR-4688 | UAGGGGCAGCAGAGGACCUGGG |
| SEQ ID NO: 91 | hsa-miR-4708-3p | AGCAAGGCGGCAUCUCUCUGAU |
| SEQ ID NO: 92 | hsa-miR-4745-5p | UGAGUGGGGCUCCCGGGACGGCG |
| SEQ ID NO: 93 | hsa-miR-4763-3p | AGGCAGGGGCUGGUGCUGGGCGGG |
| SEQ ID NO: 94 | hsa-miR-4776-5p | GUGGACCAGGAUGGCAAGGGCU |
| SEQ ID NO: 95 | hsa-miR-4787-5p | GCGGGGGUGGCGGCGGCAUCCC |
| SEQ ID NO: 96 | hsa-miR-491-5p | AGUGGGGAACCCUUCCAUGAGG |
| SEQ ID NO: 97 | hsa-mir-494-3p | UGAAACAUACACGGGAAACCUC |
| SEQ ID NO: 98 | hsa-miR-500a-3p | AUGCACCUGGGCAAGGAUUCUG |
| SEQ ID NO: 99 | hsa-miR-500a-5p | UAAUCCUUGCUACCUGGGUGAGA |
| SEQ ID NO: 100 | hsa-miR-501-3p | AAUGCACCCGGGCAAGGAUUCU |
| SEQ ID NO: 101 | hsa-miR-502-3p | AAUGCACCUGGGCAAGGAUUCA |
| SEQ ID NO: 102 | hsa-miR-532-3p | CCUCCCACACCCAAGGCUUGCA |
| SEQ ID NO: 103 | hsa-miR-532-5p | CAUGCCUUGAGUGUAGGACCGU |
| SEQ ID NO: 104 | hsa-miR-548aa | AAAAACCACAAUUACUUUUGCACCA |
| SEQ ID NO: 105 | hsa-miR-574-5p | UGAGUGUGUGUGUGUGAGUGUGU |
| SEQ ID NO: 106 | hsa-miR-629-5P | UGGGUUUACGUUGGGAGAACU |
| SEQ ID NO: 107 | hsa-miR-638 | AGGGAUCGCGGGCGGGUGGCGGCCU |
| SEQ ID NO: 108 | hsa-miR-660-5p | UACCCAUUGCAUAUCGGAGUUG |
| SEQ ID NO: 109 | hsa-miR-664a-5p | ACUGGCUAGGGAAAAUGAUUGGAU |
| SEQ ID NO: 110 | hsa-miR-708-5p | AAGGAGCUUACAAUCUAGCUGGG |
| SEQ ID NO: 111 | hsa-miR-874-3p | CUGCCCUGGCCCGAGGGACCGA |
| SEQ ID NO: 112 | hsa-miR-93-3p | ACUGCUGAGCUAGCACUUCCCG |
| SEQ ID NO: 113 | hsa-miR-99b-3p | CAAGCUCGUGUCUGUGGGUCCG |
| SEQ ID NO: 114 | SNORD113-4 | TGGACCAATGATGAGTACCATGGGGTATCT |
| | | GAAACAGGATTTTTGATTAAACCCATATGC |
| | | AATTCTGAGGTCCA |
| SEQ ID NO: 115 | SNORD114-14 | TGGACCAATGATGACAACTGCCGGCGTATG |
| | | AGTGTTGGGTGATGAATAATACGTGTCTAG |
| | | AACTCTGAGGTCCA |
| SEQ ID NO: 116 | SNORD114-3 | TGGACCAATGATGACCACTGGTGGCGTTTG |
| | | AGTCATGGACGATGAATACTACGTGTCTGA |
| | | AACTCTGAGGTCCA |
| SEQ ID NO: 117 | SNORD88A | CCGGGGCCTCCATGATGTCCAGCACTGGGC |
| | | TCCGACTGCCACTGAGGACACGGTGCCCCC |
| | | CGGGACCTTTGACACCCGGGGGTCTGAGGG |
| | | GCCCTGG |
| SEQ ID NO: 118 | SNORD88B | TTGGGGACCCCGTGATGTCCAGCACTGGGC |
| | | TCTGACTGCCCCTGAGGACACGGTGCACCC |
| | | CGGGACCTTTGACATCCGGGGTTCTGAGGG |
| | | GCCCCAC |
| SEQ ID NO: 119 | SNORD88C | CTGGGGCTCCCATGATGTCCAGCACTGGGC |
| | | TCTGATCACCCCTGAGGACACAGTGCACCC |
| | | CAGGACCTTTGACACCTGGGGGTCTGAGGG |
| | | GCCCCAG |
| SEQ ID NO: 120 | SNORD69 | AATGTGAAGCAAATGATGATAAACTGGATC |
| | | TGACTGACTGTGCTGAGTCTGTTCAATCCA |
| | | ACCCTGAGCTTCATGTT |
| SEQ ID NO: 121 | SNORD87 | ACAATGATGACTTAAATTACTTTTTGCCGTT |
| | | TACCCAGCTGAGGTTGTCTTTGAAGAAATA |
| | | ATTTTAAGACTGAGA |
| SEQ ID NO: 122 | SNORD89 | ACTGAGGAATGATGACAAGAAAAGGCCGA |
| | | ATTGCAGTGTCTCCATCAGCAGTTTGCTCTC |
| | | CATGGGCACACGATGACAAAATATCCTGAA |
| | | GCGAACCACTAGTCTGACCTCAGT |
| SEQ ID NO: 123 | SNORD92 | TGGTGCTGTGATGATGCCTTAATATTGTGGT |
| | | TTCGACTCACTGAGAGTAAAATGAGGACCT |
| | | ACAATTCCTTGGCTGTGTCTGAGCACCC |
| SEQ ID NO: 124 | SNORD110 | TTGCAGTGATGACTTGCGAATCAAATCTGT |
| | | CAATCCCCTGAGTGCAATCACTGATGTCTC |
| | | CATGTCTCTGAGCAA |
| SEQ ID NO: 125 | SNORD116-26 | TGGATCGATGATGACTATAAAAAAAATGGA |
| | | TCTCATCGGAATCTGAACAAAATGAGTGAC |
| | | CAAATCATTTCTGTGCCACTTCTGTGAGCTG |
| | | AGGTCCA |
| SEQ ID NO: 126 | SNORD116-6 | TGGATCGATGATGAGTCCTCCAAAAAAAAC |
| | | ATTCCTTGGAAAAGCTGAACAAAATGAGTG |
| | | AAAACTCATACCGTCATTCTCATCGGAACT |
| | | GAGGTCCA |
| SEQ ID NO: 127 | SNORD123 | GGTGAAAATGATGAATTCTGGGGCGCTGAT |
| | | TCATGTGACTTGAAAAATGCCATCCATTTC |
| | | CTGATTCACC |
| SEQ ID NO: 128 | SNORD105B | CCACATGCGGCTGATGACAGCACTTCTGCT |
| | | GAGACGCTGTGATTGCTCTGTCCAAAGTAA |
| | | ACGCCCTGACGCACTGTGG |
| SEQ ID NO: 129 | SNORD15A | CTTCGATGAAGAGATGATGACGAGTCTGAC |
| | | TTGGGGATGTTCTCTTTGCCCAGGTGGCCTA |
| | | CTCTGTGCTGCGTTCTGTGGCACAGTTTAAA |
| | | GAGCCCTGGTTGAAGTAATTTCCTAAAGAT |
| | | GACTTAGAGGCATTTGTCTGAGAAGG |
| SEQ ID NO: 130 | SNORD15B | CTTCAGTGATGACACGATGACGAGTCAGAA |
| | | AGGTCACGTCCTGCTCTTGGTCCTTGTCAGT |
| | | GCCATGTTCTGTGGTGCTGTGCACGAGTTC |
| | | CTTTGGCAGAAGTGTCCTATTTATTGATCGA |
| | | TTTAGAGGCATTTGTCTGAGAAGG |
| SEQ ID NO: 131 | SNORD21 | GCTGAATGATGATATCCCACTAACTGAGCA |
| | | GTCAGTAGTTGGTCCTTTGGTTGCATATGAT |
| | | GCGATAATTGTTTCAAGACGGGACTGATGG |
| | | CAGC |
| SEQ ID NO: 132 | SNORD25 | TTCCTATGATGAGGACCTTTTCACAGACCT |
| | | GTACTGAGCTCCGTGAGGATAAATAACTCT |
| | | GAGGAGA |
| SEQ ID NO: 133 | SNORD27 | ACTCCATGATGAACACAAAATGACAAGCAT |
| | | ATGGCTGAACTTTCAAGTGATGTCATCTTA |
| | | CTACTGAGAAGT |
| SEQ ID NO: 134 | SNORD28 | GTCAGATGATTTGAATTGATAAGCTGATGT |
| | | TCTGTGAGGTACAAAAGTTAATAGCATGTT |
| | | AGAGTTCTGATGGCA |
| SEQ ID NO: 135 | SNORD29 | TTTCTATGATGAATCAAACTAGCTCACTAT |
| | | GACCGACAGTGAAAATACATGAACACCTG |
| | | AGAAAC |
| SEQ ID NO: 136 | SNORD3B-2 | AAGACTATACTTTCAGGGATCATTTCTATA |
| | | GTGTGTTACTAGAGAAGTTTCTCTGAACGT |
| | | GTAGAGCACCGAAAACCCCGAGGAAGAGA |
| | | GGTAGCGTTTTCTCCTGAGCGTGAAGCCGG |
| | | CTTTCTGGCGTTGCTTGGCTGCAACTGCCGT |
| | | CAGCCATTGATGATCGTTCTTCTCTCCGTAT |
| | | TGGGGAGTGAGAGGGAGAGAACGCGGTCT |
| | | GAGTGGT |
| SEQ ID NO: 137 | SNORD3B-1 | AAGACTATACTTTCAGGGATCATTTCTATA |
| | | GTGTGTTACTAGAGAAGTTTCTCTGAACGT |
| | | GTAGAGCACCGAAAACCCCGAGGAAGAGA |
| | | GGTAGCGTTTTCTCCTGAGCGTGAAGCCGG |
| | | CTTTCTGGCGTTGCTTGGCTGCAACTGCCGT |
| | | CAGCCATTGATGATCGTTCTTCTCTCCGTAT |
| | | TGGGGAGTGAGAGGGAGAGAACGCGGTCT |
| | | GAGTGGT |
| SEQ ID NO: 138 | SNORD3D | AAGGCTATACTTTCAGGGATCATTTCTATA |
| | | GTGTGTTACTAGAGAAGTTTCTTTGAACGT |
| | | GTAGAGCACCGAAAACCCCGAGGAAGAGA |
| | | GGTAGCGTTTTCTCCTGAGCGTGAAGCCGG |
| | | CTTTCTGGCGTTGCTTGGCTGCAACTGCCGT |
| | | CAGCCATTGATGATCGTTCTTCTCTCCGTAT |
| | | TGGGGAGTGAGAGGGAGAGAACGCGGTCT |
| | | GAGTGGT |
| SEQ ID NO: 139 | SNORD30 | GTTTGTGATGACTTACATGGAATCTCGTTCG |
| | | GCTGATGACTTGCTGTTGAGACTCTGAAAT |
| | | CTGATTTTC |
| SEQ ID NO: 140 | SNORD31 | CTCACCAGTGATGAGTTGAATACCGCCCCA |
| | | GTCTGATCAATGTGTGACTGAAAGGTATTT |
| | | TCTGAGCTGTG |
| SEQ ID NO: 141 | SNORD32A | GTCAGTGATGAGCAACATTCACCATCTTTC |
| | | GTTTGAGTCTCACGGCCATGAGATCAACCC |
| | | CATGCACCGCTCTGAGA |
| SEQ ID NO: 142 | SNORD33 | GGCCGGTGATGAGAACTTCTCCCACTCACA |
| | | TTCGAGTTTCCCGACCATGAGATGACTCCA |
| | | CATGCACTACCATCTGAGGCCAC |
| SEQ ID NO: 143 | SNORD35A | GGCAGATGATGTCCTTATCTCACGATGGTC |
| | | TGCGGATGTCCCTGTGGGAATGGCGACAAT |
| | | GCCAATGGCTTAGCTGATGCCAGGAG |
| SEQ ID NO: 144 | SNORD36B | GTTGCAGTGATGTAAAATTTCTTGGCCTGA |
| | | AATTACTGTGAAGAGTAAAACCGAGCTTTT |
| | | TAACACTGAGT |
| SEQ ID NO: 145 | SNORD38A | TTCTCGTGATGAAAACTCTGTCCAGTTCTGC |
| | | TACTGAAGGGAGAGAGATGAGAGCCTTTTA |
| | | GGCTGAGGAA |
| SEQ ID NO: 146 | SNORD38B | TCTCAGTGATGAAAACTTTGTCCAGTTCTGC |
| | | TACTGACAGTAAGTGAAGATAAAGTGTGTC |
| | | TGAGGAGA |
| SEQ ID NO: 147 | SNORD41 | TGGGAAGTGATGACACCTGTGACTGTTGAT |
| | | GTGGAACTGATTTATCGCGTATTCGTACTG |
| | | GCTGATCCTG |
| SEQ ID NO: 148 | SNORD42A | AATGATGGAAAAATCATTATTGGAAAAGA |
| | | ATGACATGAACAAAGGAACCACTGAAGTG |
| SEQ ID NO: 149 | SNORD44 | CCTGGATGATGATAAGCAAATGCTGACTGA |
| | | ACATGAAGGTCTTAATTAGCTCTAACTGAC |
| | | TAA |
| SEQ ID NO: 150 | SNORD46 | GTAGGGTGATGAAAAAGAATCCTTAGGCGT |
| | | GGTTGTGGCCGTCTTGGTCACCTGTGTGCC |
| | | ACTTGCCAATGCAAGGACTTGTCATAGTTA |
| | | CACTGACT |
| SEQ ID NO: 151 | SNORD48 | AGTGATGATGACCCCAGGTAACTCTTGAGT |
| | | GTGTCGCTGATGCCATCACCGCAGCGCTCT |
| | | GACC |
| SEQ ID NO: 152 | SNORD49A | TGCTCTGATGAAATCACTAATAGGAAGTGC |
| | | CGTCAGAAGCGATAACTGACGAAGACTACT |
| | | CCTGTCTGATT |
| SEQ ID NO: 153 | SNORD56 | CCACAATGATGGCAATATTTTTCGTCAACA |
| | | GCAGTTCACCTAGTGAGTGTTGAGACTCTG |
| | | GGTCTGAGTGA |
| SEQ ID NO: 154 | SNORD59A | CCTTCTATGATGATTTTATCAAAATGACTTT |
| | | CGTTCTTCTGAGTTTGCTGAAGCCACATTTA |
| | | GGTACTGAGAAGG |
| SEQ ID NO: 155 | SNORD59B | TATTCCTCACTGATGAGTACGTTCTGACTTT |
| | | CGTTCTTCTGAGTTTGCTGAAGCCAGATGC |
| | | AATTTCTGAGAAGG |
| SEQ ID NO: 156 | SNORD73A | AATAAGTGATGAAAAAAGTTTCGGTCCCAG |
| | | ATGATGGCCAGTGATAACAACATTTTTCTG |
| | | ATGTT |
| SEQ ID NO: 157 | SNORD74 | CTGCCTCTGATGAAGCCTGTGTTGGTAGGG |
| | | ACATCTGAGAGTAATGATGAATGCCAACCG |
| | | CTCTGATGGTGG |
| SEQ ID NO: 158 | SNORD75 | AGCCTGTGATGCTTTAAGAGTAGTGGACAG |
| | | AAGGGATTTCTGAAATTCTATTCTGAGGCT |
| SEQ ID NO: 159 | SNORD76 | GCCACAATGATGACAGTTTATTTGCTACTCT |
| | | TGAGTGCTAGAATGATGAGGATCTTAACCA |
| | | CCATTATCTTAACTGAGGC |
| SEQ ID NO: 160 | SNORD78 | GTGTAATGATGTTGATCAAATGTCTGACCT |
| | | GAAATGAGCATGTAGACAAAGGTAACACT |
| | | GAAGAA |
| SEQ ID NO: 161 | SNORD83A | GCTGTTCGTTGATGAGGCTCAGAGTGAGCG |
| | | CTGGGTACAGCGCCCGAATCGGACAGTGTA |
| | | GAACCATTCTCTACTGCCTTCCTTCTGAGAA |
| | | CAGC |
| SEQ ID NO: 162 | SNORD96A | CCTGGTGATGACAGATGGCATTGTCAGCCA |
| | | ATCCCCAAGTGGGAGTGAGGACATGTCCTG |
| | | CAATTCTGAAGG |
| SEQ ID NO: 163 | SNORD4A | GGTGCAGATGATGACACTGTAAAGCGACCA |
| | | AAGTCTGAACAAAGTGATTGGTACCTCGTT |
| | | GTCTGATGCACC |
| SEQ ID NO: 164 | SNORD6 | GATGTTATGATGATGGGCGAAATGTTCAAC |
| | | TGCTCTGAAGGGGCTGAATGAAAATGGCCT |
| | | TTCTGAACATC |
| SEQ ID NO: 165 | SNORD2 | AAGTGAAATGATGGCAATCATCTTTCGGGA |
| | | CTGACCTGAAATGAAGAGAATACTCATTGC |
| | | TGATCACTTG |
| SEQ ID NO: 166 | SNORA10 | GGTCTCTCAGCTCCGCTTAACCACACGGGT |
| | | CCAGTGTGTGCTTGGCGTGTTTTCAGGGAG |
| | | GCAGAGAAAGGCTCTCCTAATGCACGACAG |
| | | ACCCGCCCAGAATGGCCTCTCTGTTCCTAG |
| | | GAGTGCGACAATT |
| SEQ ID NO: 167 | SNORA18 | GTTGAGGTCTATCCCGATGGGGCTTTTCCTG |
| | | TAGCCTGCACATCGTTGGAAACGCCTCATA |
| | | GAGTAACTCTGTGGTTTTACTTTACTCACAG |
| | | GACTATTGTTAGATCTGTGGGAAGGAATTA |
| | | CAAGACAGTT |
| SEQ ID NO: 168 | SNORA20 | CTTCCCATTTATTTGCTGCTTGTAGTCTCAC |
| | | AGTGATACGAGCAGTTATACGCATGGGATA |
| | | AAATAACATTGGGCCACTGTAAATTGAGAT |
| | | GAAGTAACCATTTTCATCTCTTCTGCAGGG |
| | | ACTAGACATTG |
| SEQ ID NO: 169 | SNORA21 | CCCCCTTTTAAAAGCACTCAATGGGCCTGT |
| | | GGCTAATGACCTATTGAGCCGTCAAGAAAG |
| | | GGGAGAGTGAAAACATCGCTTTTGGGTGAA |
| | | GTGGCAACATGTGTTGTTTGCTTCAATCGGT |
| | | GGTGTGACAAGG |
| SEQ ID NO: 170 | SNORA33 | AAGCCAGCCAATGAATCTGCTTACCTGATT |
| | | GTGTTTGTGCAGACATACTTTAAAAACTGG |
| | | CAATAGTAAAGCCATGTTACGAGCCTTAAG |
| | | GACATTGAAGTCGTTAAGGTCCCTGAGAAT |
| | | GGCTATAACAAAT |
| SEQ ID NO: 171 | SNORA2C | GTGGCCCTGACTGAAGACCAGCAGTTGTAC |
| | | TGTGGCTGTTGGTTTCAAGCAGAGGCCTAA |
| | | AGGACTGTCTTCCTGTGGTCTGTTGGCTGTT |
| | | CTGGGACCTCAGTAGGGAATGGCTATTTCA |
| | | TTTGGAAGAAACAACC |
| SEQ ID NO: 172 | SNORA3A | ATCGAGGCTAGAGTCACGCTTGGGTATCGG |
| | | CTATTGCCTGAGTGTGCTAGAGTCCTCGAA |
| | | GAGTAACTGCTGACCTTATTCACTGGCTGT |
| | | GGGCCTTATGGCACAGTCAGTCACCAGGTT |
| | | AGAGACATGC |
| SEQ ID NO: 173 | SNORA80E | TGGTAATGGATTTATGGTGGGTCCTTCTCTG |
| | | TGGGCCTCTCATAGTGTACCCATGCCATAG |
| | | CAAATGGCAGCCTCGAACCATTGCCCAGTC |
| | | CCCTTACCTGTGGGCTGTGAGCACTGAAGG |
| | | GGGTTGCACAGTG |
| SEQ ID NO: 174 | SNORA44 | CAGCATGTTTCCAAGGGCTGTGGCTGGTCA |
| | | TAGCCATGGGATCTCCAACTGCATGCAAGA |
| | | GCAACCTGGAAAGACTTTGACAGCGCAGGT |
| | | CAGTACAATACCTGCAAGCTGCCACTCAGC |
| | | TTTCCTATAATG |
| SEQ ID NO: 175 | SNORA48 | TGTCCCTGACCTGGGTAGAGTGGCATCTGG |
| | | TTGGTGATGCCCATCTCATATCAGCCAGGG |
| | | ACAAAGCAACTCCTTGTTCATCCCAGCTTG |
| | | GCTTTTGATCCGTGCCCATGCCTGGTTCATG |
| | | CCTTGGACACATAG |
| SEQ ID NO: 176 | SNORA52 | TGGTCCATCCTAATCCCTGCCGGTCCATCTG |
| | | TGGCCTGCCAGGTTTCGCTTGTGGACCAGA |
| | | GCACCCTAGAAGCCTCACCCGAGGAGTGAG |
| | | CAGGGCTCCAGTGGGCTCACGTCATGGGCA |
| | | CTTCTAGACACTC |
| SEQ ID NO: 177 | SNORA54 | GAGCACTGTTCGTAACCCGTTAGCCTGGCT |
| | | GTAGCTAATGGGTTCCATTCCGGTGCAATA |
| | | GCATTTCCAGCGACACATGACTGACTGACT |
| | | GGTGGCTTTCAGTTTCAGGTCTTGGAGACA |
| | | AAT |
| SEQ ID NO: 178 | SNORA55 | GAGCACCTGAATCTTTCCCATTCCTTGCTGC |
| | | CTCGTGCCGGTGTGGGGACAGATGGTGCTA |
| | | CAGAATGAGCAGAGGAAATCCAGACAGGT |
| | | TGTTTTCCATTTGTCTTGGGGCCTGTCTCTA |
| | | CAGCTCTGCCACATTT |
| SEQ ID NO: 179 | SNORA50C | GCGCTGTCTTTGAGCCCCCGCCGAGCTTCCT |
| | | CGTGGCGCCGGGGGTCAATCTGCAGCGCTA |
| | | GAGCATGTGCTTGCGCATAACTGGGGCCGC |
| | | CTGGCCTCCCGCGGGCGGCCTTTTTAACCG |
| | | CGAGCGACAAGA |
| SEQ ID NO: 180 | SNORA6 | TGCACACTATTAAAGCTCAGGGTGGAGGCC |
| | | AGTCTTGGCTCATGAACTTCTGAGTGTCGG |
| | | AAGTGTGCTATATCAATGGCAGGATTTTCG |
| | | CTAACACCAGTAGAGCTTGCCTCTATGACT |
| | | GGAGTTTGGTAGTACTCGCTGCCACATAG |
| SEQ ID NO: 181 | SNORA9 | TAGCAAGCCTCCAGCGTGCTTGGGTCTGCG |
| | | GTGACCCTATGCATTCCTTCAGTGCTTGCTA |
| | | GAACAGTTTTGAAACGGTTTGAGGCCTTGC |
| | | CCTGCTCCATCCAGAGCAAGGTTATAGAAA |
| | | TTTCAGACAATG |
| SEQ ID NO: 182 | SNORA73A | TCCAACGTGGATACACCCGGGAGGTCACTC |
| | | TCCCCGGGCTCTGTCCAAGTGGCGTAGGGG |
| | | AGCATAGGGCTCTGCCCCATGATGTACAAG |
| | | TCCCTTTCCACAACGTTGGAAATAAAGCTG |
| | | GGCCTCGTGTCTGCGCCTGCATATTCCTACA |
| | | GCTTCCCAGAGTCCTGTCGACAATTACTGG |
| | | GGAGACAAACCATGCAGGAAACAGCC |
| SEQ ID NO: 183 | SNORA73B | TCCAACGTGGATACCCTGGGAGGTCACTCT |
| | | CCCCAGGCTCTGTCCAAGTGGCATAGGGGA |
| | | GCTTAGGGCTCTGCCCCATGATGTACAGTC |
| | | CCTTTCCACAACGTTGAAGATGAAGCTGGG |
| | | CCTCGTGTCTGCGCCTGCATATTCCTACAGC |
| | | TTCCCAGAGTCCTGTGGACAATGACTGGGG |
| | | AGACAAACCATGCAGGAAACATAT |
| SEQ ID NO: 184 | SNORA74A | ATCCAGCGGTTGTCAGCTATCCAGGCTCAT |
| | | GTGGTGCCTGTGATGGTGTTACACTGTTGG |
| | | AAGAGCAAACACTGTCTTTATTGAGGTTTG |
| | | GCTCCAAGCACTGTTTTGGTGTTGTAGCTG |
| | | AGTACCTTTGGGCAGTGTTTTGCACCTCTGA |
| | | GAGTGGAATGACTCCTGTGGAGTTGATCCT |
| | | AGTCTGGGTGCAAACAATT |
| SEQ ID NO: 185 | SNORA75 | GTCTTCTCATTGAGCTCCTTTCTGTCTATCA |
| | | GTGGCAGTTTATGGATTCGCACGAGAAGAA |
| | | GAGAGAATTCACAGAACTAGCATTATTTTA |
| | | CCTTCTGTCTTTACAGAGGTATATTTAGCTG |
| | | TATTGTGAGACATTC |
| SEQ ID NO: 186 | SNORA64 | ACTCTCTCGGCTCTGCATAGTTGCACTTGGC |
| | | TTCACCCGTGTGACTTTCGTAACGGGGAGA |
| | | GAGAGAAAAGATCTCCTCAGGACCTCGGAT |
| | | GGGCCTTACTGTGGCCTCTCTTTCCTTGAGG |
| | | GGTGCAACAGGC |
| SEQ ID NO: 187 | SNORA66 | GTGCAAACTCGATCACTAGCTCTGCGTGAT |
| | | GTGGCAGAAGCGAAGGGAACCAGGTTTGC |
| | | AAAAGTAACTGTGGTGATGGAAATGTGTTA |
| | | GCCTCAGACACTACTGAGGTGGTTCTTTCT |
| | | ATCCTAGTACAGTC |
| SEQ ID NO: 188 | SNORA68 | ATTGCACCTAAACCCAAGAATCACTGTTTC |
| | | TTATAGCGGTGGTTTAAACAGAGGTGCAAA |
| | | CAGCAAGCGGATCTTGTCGCCTTTGGGGGG |
| | | CTGTGGCCGTGCCCCTCAAAGTGAATTTGG |
| | | AGGTTCCACAACT |
| SEQ ID NO: 189 | SNORA71D | CACCTGTATTCGAAAGTGATCGTGGGCTGC |
| | | CTGTGCCCTGGTCATTGATAGTGCAGGGAA |
| | | AGAAATCGCGGAAAGTGCTTCCCCGTGTTT |
| | | GGAGGGTCCGCTCCTGTCCCTTTCAAACTCT |
| | | GGAGCTTTCTCACACCT |
| SEQ ID NO: 190 | SCARNA17 | AGAGGCTTGGGCCGCCGAGCTGGACCCGG |
| | | ACCGGTTTTGGGTACTGTACTGGGGGCAGG |
| | | GCAGAGAGGTGGGCGGCAGTTGGGGTGCG |
| | | GTGATTGTAGTAGGCTAGGGCGCTTTCGGG |
| | | TCCCCATTGCAGCCCCCGGATGAGCCCGCA |
| | | GTATTTTCCTTATATGATCAGGTCCCATTGC |
| | | GGGCGGCGCCGCTTGCCCGGAGCCTGAGAG |
| | | GATTATGAAAACGTGGCGAGCGAAATGGG |
| | | GCCAGGGGACCTGGAGCAGGGGCGTGAGG |
| | | AGAGTAGGCAGCGGGTGAGGCTGGACGGG |
| | | AGGGAGGTCTAGGGAGGCCTCTGCCGCGG |
| | | GCACTGTGAGTCCTGGCCGATGATGACGAG |
| | | ACCACTGCGCAATCTGAGTTCTGGGAACCA |
| | | GGTGATGGAGTATGTTCTGAGAACAGACTG |
| | | AGGCCG |
| SEQ ID NO: 191 | ENSG00000201009 | TGCTGGAGTGATGAAAAAGTATCTTCAGGT |
| | | GTGGCTGTGGCCACCTTGGCCACCTGTGTG |
| | | TCACTTGCCAATGCAAGGACTTGTCATAGT |
| | | TACACTGACTGTTA |
| SEQ ID NO: 192 | ENSG00000201042 | CCCTCCTACAAAGGCATGTCTATAATTCCTT |
| | | GTCTTTGGACATGTAAGAATTGGAGGGACA |
| | | GAAATGTGGACTTGGAGAAATCTGGGGCCA |
| | | GCTTTCTCATCACAGGCTCAACATCAACCA |
| | | TGCCACATAG |
| SEQ ID NO: 193 | ENSG00000202498 | AGATCATTGATGACTTCCATATATCCATTCC |
| | | TTGGAAAGCTGAACAACATGAGTGAAAACT |
| | | CTACTGAAAAAAGAAAAGAAATGGGAGGC |
| | | CG |
| SEQ ID NO: 194 | ENSG00000206903 | CTCCATGTATCTTTGGGACCTGTCAAGTGTG |
| | | GCAGTCTCCCTTCCTTGCCATGGAAGAGCA |
| | | TATTCTTGTTTACCAGCAAAGCTGTCACCAT |
| | | TTAATTGGTATCAGATTCTGACTTGCACAA |
| | | GTAACATTC |
| SEQ ID NO: 195 | ENSG00000206913 | GAGCTTCCAGGATCACCCCTGCAGAGTGGC |
| | | TAATATTCTGCCAGCTTCGGAAAGGGAGGG |
| | | GAAGCAAGCCTGGCAGAGGCACCCATTCCA |
| | | TTCCCAGCTTGCTTAGTAGCTGGCCATGGG |
| | | AAGACACTGTGCAACACTG |
| SEQ ID NO: 196 | ENSG00000207187 | GGTCTCTCAGCTCTGCTTAACCACACGGGT |
| | | CCAGTGTGTGCTTGGCGTGTTTTCAGGGAG |
| | | GCAGAGAAAGGCTCTCCTAATGCACGACAG |
| | | ACCCGCCCAGAATGGCCTCTCTGTTCCTAG |
| | | GAGTGTGACAATT |
| SEQ ID NO: 197 | ENSG00000212378 | ATGTAATAATGTTCATCAAATGTCTGACCT |
| | | GAAATGAGCATGTAGACAAGTTAATTTAAC |
| | | ACTGAAGAA |
| SEQ ID NO: 198 | ENSG00000212587 | TGCACTTATGTATGTTTTTGTTTAACTTGTG |
| | | GACAAAGACTTTAGGAAAGGTGCAAAAAA |
| | | TAAATCTTCTTTTGCAACCCAGAACTCATTG |
| | | TTCAGTATGAGTTTTGATACATATCAGAAT |
| | | GGATACT |
| SEQ ID NO: 199 | ENSG00000221060 | AGGGTTTGCTTAGGGCAGGGAGGTTGAAGA |
| | | GTGGCTCCTCTGTTTACAATACACCAAACA |
| | | GGAATCTGGGGTCATTGTGACAAGGGGCAC |
| | | AAAACTTGTGTCCTCCCTACATGTGAAAAA |
| | | AAAAAAAGA |
| SEQ ID NO: 200 | ENSG00000221252 | AAGACTGTACTTTCAGGAATCATTTCTATA |
| | | GTTCATTACTAGAGAAATTTCTCTGAACAT |
| | | GTAGAGCACCAGAAAATATTTTTAAAGATT |
| | | TCTTTAGGCTGGGCGTGGTGGCTCACGCCT |
| | | GTAATCCCAGCACTTTGGGAGGCCGAAGTG |
| | | GGCGGATCATCTGAGGTCGGGAGTTCGAGA |
| | | CCAGCCTGACTAACATG |
| SEQ ID NO: 201 | ENSG00000238422 | ATCCTTTTGTAGTTCTTAAGTGTGATGATTG |
| | | GGTTTTCATGCTTATGTGTGAAATGTGCCTT |
| | | TCTCAAACCTTGTTATGACACTGGCACATT |
| | | ACCTGTGTGACG |
| SEQ ID NO: 202 | ENSG00000238549 | AAATTTTAGGAGTACCTAAGTGTGATGATT |
| | | TGGTTTTCACATTCATGTGTGAGCTGTGCCT |
| | | GCCTTTTGTTACAAGGGCATATTACCCTTTG |
| | | TTGTGAAA |
| SEQ ID NO: 203 | ENSG00000238956 | ACTGACCTGAAATGAAGAGAATACTCATTG |
| | | CTGA |
| SEQ ID NO: 204 | ENSG00000239054 | GTCCACTTGTAGTTCATAAGCAACATGATT |
| | | TGGTTTTCATGCTGATGTGTGAGATGTGCCT |
| | | CCCTCAAACCTTGTTACTATGTTGGCACATT |
| | | ACAAGTTTGACA |
| SEQ ID NO: 205 | ENSG00000239055 | ATCCTTTCGTAGTTTATAAGAGTGATGATTA |
| | | GGTCTTCATGCTCATGTGTGAAATGTGCCTC |
| | | CCTCAAACCATGTTAGGACGTTGGCATATT |
| | | GCCCATCTGAAA |
| SEQ ID NO: 206 | ENSG00000239154 | ATCCTTTTGTAGTTTATGAGCATGATGACTG |
| | | GGTTTTCACAGGTATGTGTGAGATGTGCCA |
| | | TCCTCGAACCTTGTTATGATGTCGGCATATT |
| | | GTCAGTCTGACA |
| SEQ ID NO: 207 | ENSG00000251838 | CTTCTGCTAAGGTTTACACTATAGATGCAG |
| | | GAAAAAAAATGTCCTCACACTGTCTGTCTG |
| | | ATTGTGGCAGCTGAGATTGAATAGAGAAAT |
| | | ATAGGG |
| SEQ ID NO: 208 | ENSG00000252277 | GGATTGACGATGACTTTAAAAAAAAAAAAT |
| | | CTCATTGAAATCTGAAAAAAATGAGTGACC |
| | | AAACCACTTCTGTGAGCTGAGGTCC |
| SEQ ID NO: 209 | ENSG00000252921 | AAGACTATACTTTCAGGGATCATTTCTATA |
| | | GTTCTTTACTAGAGAAGTTTCTCTGAACATG |
| | | TAGAGCACTGTGCCTTAAAAAAGAAAAAA |
| | | AAAAGGGCTGGGCATGGTGGCTCACGCCTG |
| | | TAATCCCAGCACTTTGGGAGG |

1. A method for diagnosing indolent or aggressive prostate cancer in a subject, comprising:
a) obtaining a biological sample from a human patient;
b) detecting the expression level of at least 10 ncRNAs selected from the group consisting of SEQ ID NOs:1-209 by contacting the biological sample with a reagent in an in vitro assay; and
c) identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample.

2. The method according to claim 1, wherein the biological sample is selected from the group consisting of prostate tissue and prostate cells.

3. The method according to claim 2, wherein the prostate tissue is formalin-fixed paraffin-embedded tissue.

4. The method of claim 2, further comprising extracting ncRNA from prostate tissue or prostate cells.

5. The method of claim 1, wherein the detecting is done by the method selected from the group consisting of reverse transcription polymerase chain reaction, polymerase chain reaction, and nucleic acid hybridization, or any combination thereof.

6. The method of claim 1, wherein the reagent is selected from the group consisting of oligoribonucleotide primers, oligonucleotide primers, oligoribonucleotide probes, and oligonucleotide probes, or any combination thereof.

7. The method according to claim 1, wherein ncRNAs are selected from the group consisting of miRNA, C/D box snoRNA, H/ACA box snoRNA, scaRNAs, piRNAs, and lncRNAs.

8. The method of claim 4, wherein the prostate tissue sample is analyzed for the relative and cumulative expression profile for at least 10 ncRNAs selected from the group consisting of SEQ ID NOs:21, 27, 33, 55, 61, 67, 71, 86, 94, 95, 102, 105, 111, 112, 126, 131, 136, 141, 160, 162, 166, 185, 189, 193, and 202, and compared to the relative and cumulative expression profile for the same ncRNAs in a prostate tissue sample with indolent cancer or a prostate tissue sample with aggressive cancer.

9. A method of screening a subject for indolent or aggressive prostate cancer, comprising:
a) hybridizing ncRNAs from a biological sample from the subject with a microarray comprising probes for whole-genome ncRNAs;
b) detecting the relative abundance of hybridization products for at least 10 ncRNAs selected from the group consisting of SEQ ID NOs:1-209; and
c) comparing the cumulative expression levels of the at least 10 ncRNAs from the biological sample with the cumulative expression levels of the at least 10 ncRNAs from an indolent prostate cancer biological sample, wherein an increased level of expression of the at least 10 ncRNAs in the subject is indicative of aggressive prostate cancer in further need of treatment, and wherein an equal or less than level of expression of the at least 10 ncRNAs in the subject is indicative of indolent prostate cancer not in need of further treatment.

10. The method according to claim 9, wherein the biological sample is selected from the group consisting of prostate tissue and prostate cells.

11. The method according to claim 10, wherein the prostate tissue is formalin-fixed paraffin-embedded prostate tissue.

12. The method of claim 10, further comprising extracting ncRNA from prostate tissue or prostate cells.

13. The method of claim 9, wherein the detecting is done by the method selected from the group consisting of reverse transcription polymerase chain reaction, polymerase chain reaction, and nucleic acid hybridization, or any combination thereof.

14. The method of claim 9, wherein the reagent is selected from the group consisting of oligoribonucleotide primers, oligonucleotide primers, oligoribonucleotide probes, and oligonucleotide probes, or any combination thereof.

15. The method according to claim 9, wherein ncRNAs are selected from the group consisting of miRNA, C/D box snoRNA, H/ACA box snoRNA, scaRNAs, piRNAs, and lncRNAs.

16. The method of claim 9, wherein the prostate tissue sample is analyzed for the relative and cumulative expression profile for at least 10 ncRNAs selected from the group consisting of SEQ ID NOs:21, 27, 33, 55, 61, 67, 71, 86, 94, 95, 102, 105, 111, 112, 126, 131, 136, 141, 160, 162, 166, 185, 189, 193, and 202, and compared to the relative and cumulative expression profile for the same ncRNAs in a prostate tissue sample with indolent cancer or a prostate tissue sample with aggressive cancer.

17. A method of treatment of aggressive prostate cancer in a subject, comprising:
a) obtaining a biological sample from a human patient;
b) detecting the expression level of at least 10 ncRNAs selected from the group consisting of SEQ ID NOs:1-209 by contacting the biological sample with a reagent in an in vitro assay;
c) identifying the subject as having aggressive prostate cancer when the combined expression level of the at least 10 ncRNAs is higher than the combined expression level in an indolent prostate cancer biological sample, or identifying the subject as having indolent prostate cancer when the combined expression level of the at least 10 ncRNAs is less than or equal to the combined expression level in an indolent prostate cancer biological sample; and
d) treating the aggressive prostate cancer by one or more of:
i. surgery for partial or complete surgical removal of prostate tissue;
ii. administering an effective dose of radiation; and
iii. administering a therapeutically effective amount of a medication for the treatment of aggressive prostate cancer.

18. The method according to claim 17, wherein the surgery is chosen from laparoscopic surgery, laparoscopic radical prostatectomy, prostatectomy, and radical retropubic prostatectomy.

19. The method according to claim 17, wherein the radiation is chosen from external beam radiotherapy, brachytherapy, 3D conformational therapy, gamma knife therapy, and particle beam therapy.

20. The method according to claim 17, wherein the medication for the treatment of aggressive prostate cancer is chosen from a chemotherapeutic and a sex hormone suppressor.

21. The method according to claim 17, wherein the chemotherapeutic is chosen from docetaxel (Taxotere), cabazitaxel (Jetvana), Goserelin (Zoladex), Flutamide (Eulexin), Bicalutamide (Casodex), Abiraterone (Zytiga), and Nilutamide (Nilandron).

22. The method according to claim 21, wherein the chemotherapeutic is selected based on the combined expression level of the at least 10 ncRNAs.

23. The method according to claim 20, wherein the sex hormone suppressor is Leuprolide (Lupron).

24. The method of claim 17, wherein the biological sample is analyzed for the relative and cumulative expression profile for at least 10 ncRNAs selected from the group consisting of SEQ ID NOs:21, 27, 33, 55, 61, 67, 71, 86, 94, 95, 102, 105, 111, 112, 126, 131, 136, 141, 160, 162, 166, 185, 189, 193, and 202, and compared to the relative and cumulative expression profile for the same ncRNAs in a prostate tissue sample with indolent cancer or a prostate tissue sample with aggressive cancer.

25. A kit for the method as recited in claim 1, comprising:
a) a first reagent solution for isolating ncRNAs from a patient biological sample; and
b) a second reagent solution for detecting expression levels of at least 10 ncRNAs, from the first reagent solution, selected from the group consisting of SEQ ID NOs:1-209.
